(19) United States
(12) Patent Application Publication
(10) Pub. No.: US 2026/0042806 A1
(43) Pub. Date: Feb. 12, 2026

(54) NOVEL PORE MONOMERS AND PORES

(71) Applicants: Oxford Nanopore Technologies PLC, Oxford (GB); The Regents of the University of California, Oakland, CA (US)

(72) Inventors: Elizabeth Jayne Wallace, Oxford (GB); Lakmal Nishantha Jayasinghe, Oxford (GB); William F. DeGrado, Oakland, CA (US); Lee Schnaider, Oakland, CA (US); Hyunil Jo, Oakland, CA (US)

(73) Assignees: Oxford Nanopore Technologies PLC, Oxford (GB); The Regents of the University of California, Oakland, CA (US)

(21) Appl. No.: 19/101,995

(22) PCT Filed: Aug. 9, 2023
(86) PCT No.: PCT/EP2023/072106

§ 371 Date: Feb. 7, 2025

Related U.S. Application Data
(60) Provisional application No. 63/370,875, filed on Aug. 9, 2022.

(51) Int. Cl. C07K 14/245; B82Y 5/00; B82Y 15/00; G01N 33/53

(52) U.S. Cl. CPC C07K 14/245; G01N 33/5308; B82Y 5/00; B82Y 15/00; G01N 2333/245

(57) ABSTRACT

The present invention relates to novel pore monomer conjugates, pore complexes formed from the conjugates and their uses in analyte detection and characterisation.

### RELATED APPLICATIONS

This application is a national stage filing under 35 U.S.C. § 371 of international PCT application PCT/EP2023/072106, filed Aug. 9, 2023, which claims priority under 35 U.S.C. § 119(e) to U.S. provisional patent application, U.S. Ser. No. 63/370,875, filed Aug. 9, 2022, the entire contents of each of which are herein incorporated by reference.

### REFERENCE TO AN ELECTRONIC SEQUENCE LISTING

The contents of the electronic sequence listing (0036670131US01-SUBSEQ-KZM.xml; Size: 10,524 bytes; and Date of Creation: Mar. 4, 2025) are herein incorporated by reference in its entirety.

### TECHNICAL FIELD

The present invention relates to novel pore monomer conjugates, pore complexes formed from the conjugates and their uses in analyte detection and characterisation.

### BACKGROUND

Nanopore sensing is an approach to analyte detection and characterization that relies on the observation of individual binding or interaction events between the analyte molecules and an ion conducting channel. Two of the essential components of analyte characterization using nanopore sensing are (1) the control of analyte movement through the pore and (2) the discrimination of the composing building blocks as the analyte is moved through the pore. During nanopore sensing, the narrowest part of the pore forms the most discriminating part of the nanopore with respect to the current signatures as a function of the passing analyte. CsgG was identified as an ungated, non-selective protein secretion channel from Escherichia coli (Goyal et al., 2014) and has been used as a nanopore for detecting and characterising analytes. Mutations to the wild-type CsgG pore that improve the properties of the pore in this context have also been disclosed (WO 2016/034591, WO 2017/149316, WO 2017/149317, WO 2017/149318, WO 2018/211241, and WO 2019/002893, all incorporated by reference herein in their entirety).

For polynucleotide analytes, nucleotide discrimination is achieved by measuring the current as the polynucleotide passes through the pore. Multiple nucleotides contribute to the observed current, so the height of the channel constriction and extent of the interaction with the polynucleotide affect the relationship between observed current and polynucleotide sequence. While the current range and signal-to-noise ratio for nucleotide discrimination have been improved through mutation of the CsgG pore, a sequencing system would have higher performance if the current differences between nucleotides could be improved further. Accordingly, there is a need to identify novel ways to improve nanopore sensing features.

### SUMMARY OF THE INVENTION

The inventors have surprisingly shown that pore complexes formed from pore monomer conjugates in which a CsgG pore monomer is attached to a CsgF peptide by specific linkers display an increased current range and/or increased signal-to-noise ratio (SNR) during analyte characterisation. The inventors have also surprisingly shown that pore complexes formed from pore monomer conjugates in which a loop region in a CsgG pore monomer is attached to a CsgF peptide display an increased current range and increased signal-to-noise ratio (SNR) during analyte characterisation. Increased current range and increased SNR both improve the ability to discriminate analytes as they pass through the pore. Neither the improvement in range nor the improvement in SNR could be predicted from previous experiments using CsgG and CsgF. The invention therefore provides a pore monomer conjugate comprising a CsgG pore monomer attached to a CsgF peptide, wherein the CsgF peptide is attached to the CsgG pore monomer by a sulfonyl fluoride-containing linker, a sulfonyl triazole-containing linker, a fluorosulfate-containing linker, or fluoroacetamide-containing linker. The invention also provides a pore monomer conjugate comprising a CsgG pore monomer attached to a CsgF peptide, wherein the CsgF peptide is attached to a residue in the CsgG pore monomer corresponding to any one of positions 143, 146, 190, 192, 193, 194, 195, 196, 197, 198, 199 or 200 in SEQ ID NO: 3.

The invention also provides:
a construct comprising two or more covalently attached pore monomer conjugates of the invention;
a pore complex comprising at least one pore monomer conjugate of the invention or at least one construct of the invention, wherein the CsgF peptide(s) form(s) a constriction in the pore complex;
a pore multimer comprising two or more pores, wherein at least one of the pores is a pore complex of the invention;
a membrane comprising a pore complex of the invention or a pore multimer of the invention;
a method for producing a pore monomer conjugate of the invention comprising attaching the CsgF peptide to the cysteine residue in the CsgG pore monomer;
a method for producing a pore complex of the invention or a pore multimer of the invention, the method comprising expressing at least one pore monomer conjugate of the invention or a construct of the invention and sufficient pore monomers or constructs to form the pore complex or the pore multimer in a host cell and allowing the pore complex or the pore multimer to form in the host cell;
a method for producing a pore complex of the invention or a pore multimer of the invention, the method comprising contacting at least one pore monomer conjugate of the invention or a construct of the invention with sufficient pore monomers or constructs in vitro and allowing the formation of the pore complex or the pore multimer;
a method for determining the presence, absence or one or more characteristics of a target analyte, comprising the steps of:
(i) contacting the target analyte with a pore complex of the invention or a pore multimer of the invention, such that the target analyte moves with respect to the pore complex or the pore multimer; and
(ii) taking one or more measurements as the analyte moves with respect to the pore complex or the pore multimer and thereby determining the presence, absence or one or more characteristics of the analyte.
use of a pore complex of the invention or a pore multimer of the invention to determine the presence, absence or one or more characteristics of a target analyte;
a polynucleotide which encodes a pore monomer conjugate of the invention or a construct of the invention;
a kit for characterising a target analyte comprising (a) a pore complex of the invention or a pore multimer of the invention and (b) the components of a membrane;
a kit for characterising a target polynucleotide or a target polypeptide comprising (a) a pore complex of the invention or a pore multimer of the invention and (b) a polynucleotide binding protein;
an apparatus for characterising a target polynucleotide or a target polypeptide in a sample, comprising (a) a plurality of pore complexes of the invention or a plurality of pore multimers of the invention and (b) a plurality of polynucleotide binding proteins;
an array comprising a plurality of membranes of the invention;
a system comprising (a) a membrane of the invention or an array of the invention, (b) means for applying a potential across the membrane(s) and (c) means for detecting electrical or optical signals across the membrane(s);
an apparatus comprising a pore complex of the invention or a pore multimer of the invention inserted into an in vitro membrane; and
an apparatus produced by a method comprising (i) obtaining a pore complex of the invention or a pore multimer of the invention and (ii) contacting the pore complex or a pore multimer with an in vitro membrane such that the pore complex or the pore multimer is inserted in the in vitro membrane.

### DESCRIPTION OF THE FIGURES

FIG. 1: Generic workflow of crosslinking with a sulfonyl-fluoride linker. For example, CsgF peptide is functionalised with a sulfonyl fluoride linker on Lys30. When the functionalised CsgF is in close proximity with CsgG pore, the OH group on Y196 in CsgG pore will displace fluoride group to covalently link CsgG pore to CsgF peptide.

FIG. 2: Possible examples of functionalised CsgF peptides. A: CsgF peptide with a fluorosulfate linker. B-E: CsgF peptides with arylsulfonyl fluoride linkers. F-G: CsgF peptide with alkylsulfonyl fluoride linkers. H-L: CsgF peptides with arylsulfonyl fluoride linkers. M-N: CsgF peptides with fluoroacetamide linkers. The representative reagents and chemistries for functionalisation are as follows: Amide bond formation with 3-[(fluorosulfonyl)oxy]benzoic acid (META-OSO₂F) for A, 4-(fluorosulfonyl)phenyl]acetic acid (CH₂PH—P—SO₂F) for B, 4-(fluorosulfonyl)benzoic acid (PARA-SO₂F) for C, 3-(fluorosulfonyl)benzoic acid (META-SO₂F) for D, 4-(2-Aminoethyl)benzene-1-sulfonyl fluoride for E, 4-(2-fluoroacetamido)benzoic acid for M, 3-(2-fluoroacetamido)benzoic acid for N, 1,4-addition with ethenesulfonyl fluoride for F. Azide-alkyne cycloadditon with 2-[(prop-2-yn-1-yl)oxy]ethane-1-sulfonyl fluoride for G. Amide bond formation followed by sulfonyl triazole synthesis for H-L (as described in WO 2020/214336 herein incorporated by reference in its entirety) substituted 3-phenyl-1H-1,2,4-triazole and 4-(chlorosulfonyl)benzoyl chloride for H (X=H, OMe, CN, Br, Ph, or CF3), substituted 3-phenyl-1H-1,2,4-triazole and 3-(chlorosulfonyl)benzoyl chloride for 1 (X=H, OMe, CN, Br, Ph, or CF3.), 1H-1,2,4-triazole and 4-(chlorosulfonyl)benzoyl chloride for J, 3-(pyridin-3-yl)-1H-1,2,4-triazole and 4-(chlorosulfonyl)benzoyl chloride for K, 3-(pyridin-3-yl)-1H-1,2,4-triazole and 3-(chlorosulfonyl)benzoyl chloride for L. 1,2,4-triazole can be replaced with 1,2,3-triazole.

FIG. 3: SDS-PAGE gel analysis of the CsgG-only pore controls and CsgG/CsgF complexes when broken down to their constituent monomer components upon boiling in the presence of DTT. Lane 2 does not show a band shift compared to the CsgG-only control in lane 1. Hence, this implies that the CsgG monomers do not remain bound to the CsgF peptides upon heating in the absence of sulfonyl fluoride. Lanes 3, 4 and 6 show a band shift compared with the CsgG-only control in lanes 1, indicating a covalent linkage between CsgG and CsgF. In lane 5, much of the band is the same height as the CsgG-only control in lane 1 suggesting that the CsgF does not remain bound to CsgG, however the electrophysiology recordings demonstrate the sample is mostly complex and so it believed that the C-term CsgF-META-OSO₂F is hydrolysing upon boiling in DTT. Key:
1. CsgG-F56Q: ONLP20134
2. CsgG-F56Q/CsgF-del(S31-F119): ONLP20783
3. CsgG-F56Q/CsgF-K30-META-SO₂F-del(S31-F119): ONLP20784
4. CsgG-F56Q/CsgF-K30-CH₂—PH—P—SO₂F-del(S31-F119): ONLP20822
5. CsgG-F56Q/CsgF-K30-META-OSO₂F-del(S31-F119): ONLP20820
6. CsgG-F56Q/CsgF-K30-PARA-SO₂F-del(S31-F119): ONLP20821

FIG. 4: Bar chart showing the classification of the pores inserted into a minION flow cell. In the absence of the CsgF peptide, the majority of pores are CsgG-only pores. Note that a small number of pores get misclassified as CsgG/CsgF complexes. For the pore sample with the del(S31-F119) CsgF peptide, approximately half the of channels do not retain the CsgF peptides in the absence of any covalent attachment chemistry. However, when the pore complex comprises CsgF that is functionalized with a sulfonyl fluoride, a high proportion of CsgG/CsgF complexes are observed. The thresholds used to classify the inserted pore types are as follows: CsgG-only pores=pores with open pore current between 160 pA and 200 pA; CsgG/CsgF complexes=pores with open pore currents between 70 pA and 140 pA. Both classifications also have open pore noise<18 pA.

FIG. 5: Bar chart showing the averaged classification of pores inserted into a minION flow cell. The data is an average across runs (flow cells) of the same pores shown in FIG. 4.

FIG. 6: Snapshots of run reports showing the ionic current (pA) versus time (s) as single stranded DNA translocates through CsgG-only pores. Each individual graph corresponds to a single pore inserted into a minION flow cell. The open pore current observed for these CsgG-only pores is approximately 170-200 pA under the applied voltage of −180 mV.

FIG. 7: Snapshots of run reports showing the ionic current (pA) versus time (s) as single stranded DNA translocates through CsgG that comprises the del(S31-F119) CsgF peptides, plus/minus the sulfonyl fluoride (PARA-SO₂F). Each individual graph corresponds to a single pore inserted into a minION flow cell. The open pore current observed for the CsgG/CsgF complexes is approximately 100 pA under the applied voltage of −180 mV. Note that the pore complex without the sulfonyl fluoride is unstable and as such the CsgF peptides are not retained within CsgG for all channels e.g., channel 177 is pore only, whereas channel 179 is CsgG/CsgF complex. However, when complex contains a sulfonyl fluoride peptide variant an improvement is stability is seen and the peptide is retained within CsgG i.e channel 165 is CsgG/CsgF complex.

FIG. 8: Snapshots of run reports showing the ionic current (pA) versus time (s) as single stranded DNA translocates through CsgG that comprises the del(S31-F119) CsgF peptides, with a sulfonyl fluoride (either the META-OSO₂F or CH₂PH—P—SO₂F peptide variants). Each individual graph corresponds to a single pore inserted into a minION flow cell. The open pore current observed for the CsgG/CsgF complexes is approximately 100 pA under the applied voltage of −180 mV.

FIG. 9: Snapshots of run reports showing the ionic current (pA) versus time (s) as single stranded DNA translocates through CsgG that comprises the del(S31-F119) CsgF peptides with a sulfonyl fluoride, META-SO₂F. Each individual graph corresponds to a single pore inserted into a minION flow cell. The open pore current observed for the CsgG/CsgF complexes is approximately 100 pA under the applied voltage of −180 mV.

FIG. 10: Ionic current (pA) versus time (s) traces as single stranded DNA translocates through CsgG-only pores. The raw current trace is shown in black lines and the event detected signal is shown in red lines. For each pore, the top row shows the full DNA current trace, whilst the bottom row shows a zoomed in view of the first section of the current trace.

FIG. 11: Ionic current (pA) versus time (s) traces as single stranded DNA translocates through CsgG that comprises the del(S31-F119) CsgF peptides, plus/minus the sulfonyl fluoride (PARA-SO₂F). The raw current trace is shown in black lines and the event detected signal is shown in red lines. For each pore, the top row shows the full DNA current trace, whilst the bottom row shows a zoomed in view of the first section of the current trace.

FIG. 12: Ionic current (pA) versus time (s) traces as single stranded DNA translocates through CsgG that comprises the del(S31-F119) CsgF peptides with a sulfonyl fluoride (either the META-OSO₂F or CH₂PH—P—SO₂F peptide variants). The raw current trace is shown in black lines and the event detected signal is shown in red lines. For each pore, the top row shows the full DNA current trace, whilst the bottom row shows a zoomed in view of the first section of the current trace.

FIG. 13: Ionic current (pA) versus time (s) traces as single stranded DNA translocates through CsgG that comprises the del(S31-F119) CsgF peptides with a sulfonyl fluoride, META-SO₂F. The raw current trace is shown in black lines and the event detected signal is shown in red lines. For each pore, the top row shows the full DNA current trace, whilst the bottom row shows a zoomed in view of the first section of the current trace.

FIG. 14: Box plots showing the signal metrics of CsgG-based pores. SNR is the signal to noise ratio which is the range of the ionic current divided by the noise as single stranded DNA is translocating through the pore. The SNR increases in the presence of the sulfonyl fluoride peptide variants, e.g., the median SNR and range are higher for CsgG-WT-F56Q-Q153C/CsgF-WT-K30-META-SO₂F-del(S31-F119) than for CsgG-WT-F56Q/CsgF-WT-del(S31-F119).

FIG. 15: The structure and size of the wild-type CsgG pore from Escherichia coli strain K12 (the databank accession code for this structure is 4UV3). The distances shown are measured from backbone to backbone of the amino acids forming the pore structure. The CsgG pore is a tightly interconnected symmetrical nonameric pore that resembles a crown. The overall height is 98 Å, and the largest outer diameter is 120 Å. It defines a central channel and consists of three parts: (A) the cap region, (B) the constriction region and (C) the transmembrane beta barrel region. Cap axial length, or height, is 39 Å. It has an inner diameter of 43 Å and a 66 Å mouth. The beta barrel has 36 strands, an axial length of 39 Å and inner diameter of 55 Å. Transition between pore cap and beta barrel is sharp, being the constriction located among them, at the level of the predicted lipid-aqueous interface. The constriction is approximately 18.5 Å in diameter and exhibits a length of 20 Å along the axis of the channel.

FIG. 16. Examples of amino acid residue with reactive groups.

FIG. 17: (A) Extracellular view of the CsgG:CsgF complex (based on PDB ID: 6L7C). (B) Orientation of the CsgG:CsgF complex within the bacterial membrane. (C) Schematic of the cross link between a nucleophilic residue on CsgG and a SuTide via proximity-enhanced SuFEx reaction. (D) The Cₐₗₚₕₐ-Cₐₗₚₕₐ distances between the six C-terminal residues of CsgF₍₁₋₃₅₎ residues and the target nucleophile Tyr196 on CsgG. (E) Structure of the three SuTide warheads; 3-SO₂F-CsgF, 4-SO₂F-CsgF and 4-CH₂SO₂F—CsgF.

FIG. 18: (A) Representative SDS-PAGE gels showing the time-dependent formation of the complex between each SuTide and CsgG. The gels demonstrate a progressive decrease in the CsgG monomer (corresponding to an apparent molecular weight of 35 kDa), accompanied by a corresponding increase in the intensity of the single covalent CsgG:SuTide complex band over time (corresponding to an apparent molecular weight of 38 kDa). (B) Table detailing the T_{1/2} of the formation of each CsgG:SuTide complex.

FIG. 19: (A) Top: Structure of 3-SO₂F-CsgF with Tyr196 demonstrating the angle, q_{O,S,F} between the incoming phenolic oxygen relative to the fluoride leaving group and the distance between the incoming phenolic oxygen and sulfur d_{O,S}. Bottom: Examples of 3-SO₂F-CsgF with Tyr196 in the top clusters taken from three independent MD simulations for this probe. (B) Top: Binned radial distribution plots of d_{O,S} for each probe. Values are the mean of three independent runs. The smallest d_{O,S} observed was 3 Å. Bottom: Alignment of all Tyr196 residues from three independent MD simulations and the respective location of each sulfur atom from 3-SO₂F-CsgF. (C) Apparent molar concentrations (Cₐₚₚ) per distance bin. These were calculated from the computation of the binned radial distributions described in B to probabilities per unit volume (D) Binned q_{O,S,F} angle plots for each probe for d_{O,S} distances<4 Å. Values are the mean of three independent runs. (E) 2-dimensional plots of d_{O,S} versus q_{O,S,F} for each probe. Values are the mean of three independent runs.

### DESCRIPTION OF THE SEQUENCE LISTING

SEQ ID NO: 1 shows the polynucleotide sequence of wild-type E. coli CsgG from strain K12, including signal sequence (Gene ID: 945619).

SEQ ID NO: 2 shows the amino acid sequence of wild-type E. coli CsgG including signal sequence (Uniprot accession number POAEA2).

SEQ ID NO: 3 shows the amino acid sequence of wild-type E. coli CsgG as a mature protein (Uniprot accession number POAEA2).

SEQ ID NO: 4 shows the polynucleotide sequence of wild-type E. coli CsgF from strain K12, including signal sequence (Gene ID: 945622).

SEQ ID NO: 5 shows the amino acid sequence of wild-type E. coli CsgF including signal sequence (Uniprot accession number POAE98).

SEQ ID NO: 6 shows the amino acid sequence of wild-type E. coli CsgF as a mature protein (Uniprot accession number POAE98).

SEQ ID NO: 7 shows a synthetic construct used in Example 1.

### DETAILED DESCRIPTION

All publications, patents and patent applications cited herein, whether supra or infra, are hereby incorporated by reference in their entirety. All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the invention contained in the specification, the specification is intended to supersede and/or take precedence over any such contradictory material.

The present invention will be described with respect to particular embodiments and with reference to certain drawings but the invention is not limited thereto but only by the claims. Any reference signs in the claims shall not be construed as limiting the scope. Of course, it is to be understood that not necessarily all aspects or advantages may be achieved in accordance with any particular embodiment of the invention. Thus, for example those skilled in the art will recognize that the invention may be embodied or carried out in a manner that achieves or optimizes one advantage or group of advantages as taught herein without necessarily achieving other aspects or advantages as may be taught or suggested herein.

In addition, as used in this specification and the appended claims, the singular forms “a”, “an”, and “the” include plural referents unless the content clearly dictates otherwise. Thus, for example, reference to “a polynucleotide” includes two or more polynucleotides, reference to “a polynucleotide binding protein” includes two or more such proteins, reference to “a helicase” includes two or more helicases, reference to “a monomer” refers to two or more monomers, reference to “a pore” includes two or more pores and the like.

In all of the discussion herein, the standard one letter codes for amino acids are used. These are as follows: alanine (A), arginine (R), asparagine (N), aspartic acid (D), cysteine (C), glutamic acid (E), glutamine (Q), glycine (G), histidine (H), isoleucine (I), leucine (L), lysine (K), methionine (M), phenylalanine (F), proline (P), serine (S), threonine (T), tryptophan (W), tyrosine (Y) and valine (V). Standard substitution notation is also used, i.e., Q42R means that Q at position 42 is replaced with R.

In the paragraphs herein where different amino acids at a specific position are separated by the / symbol, the/symbol means “or”. For instance, Q87R/K means Q87R or Q87K. In the paragraphs herein where different positions are separated by the/symbol, the/symbol means “and” such that Y51/N55 is Y51 and N55.

The general definitions in WO 2019/002893 are incorporated by reference herein in their entirety.

### Pore Monomer Conjugates

The invention provides pore monomer conjugates comprising a CsgG pore monomer attached to a CsgF peptide. The CsgG pore monomer is preferably covalently attached to the CsgF peptide. Suitable CsgG pore monomers and CsgF peptides are described in more detail below.

In one embodiment, the CsgF peptide is attached to the CsgG pore monomer by a sulfonyl fluoride-containing linker, a sulfonyl triazole-containing linker, a fluorosulfate-containing linker, or fluoroacetamide-containing linker.

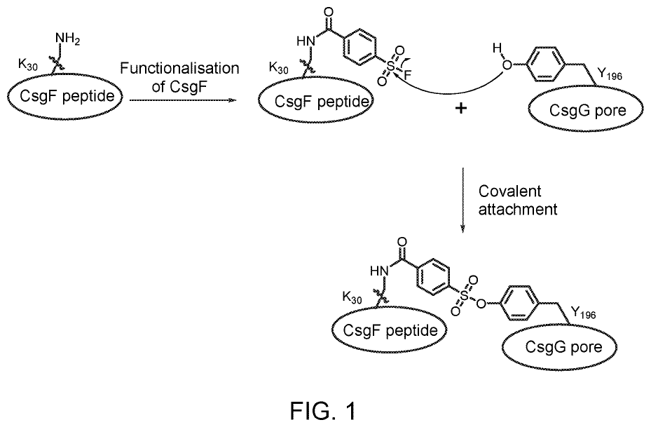

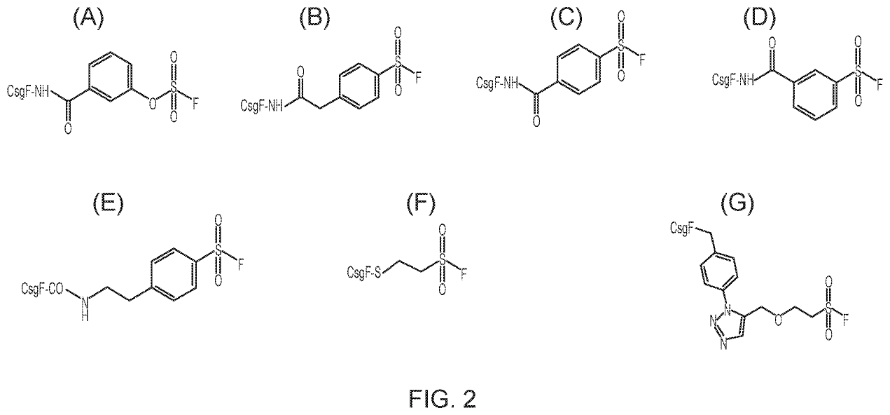

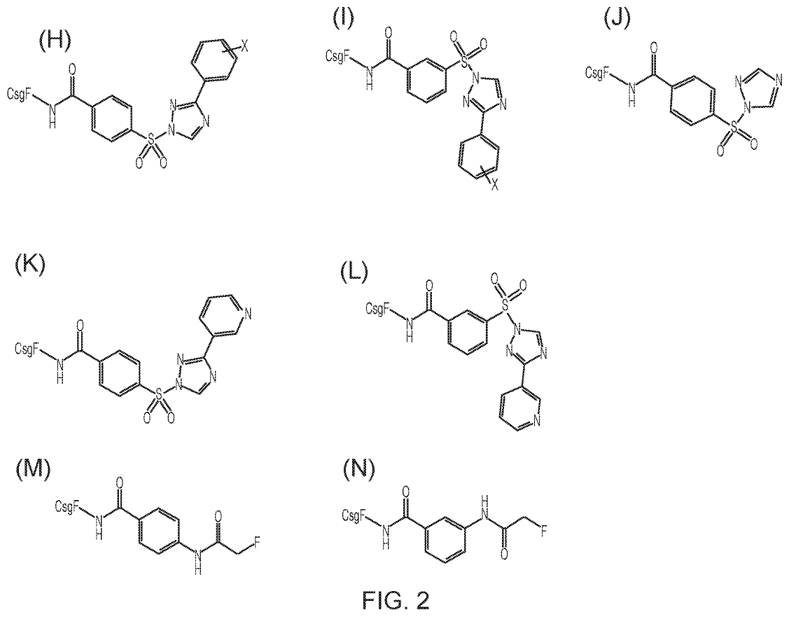

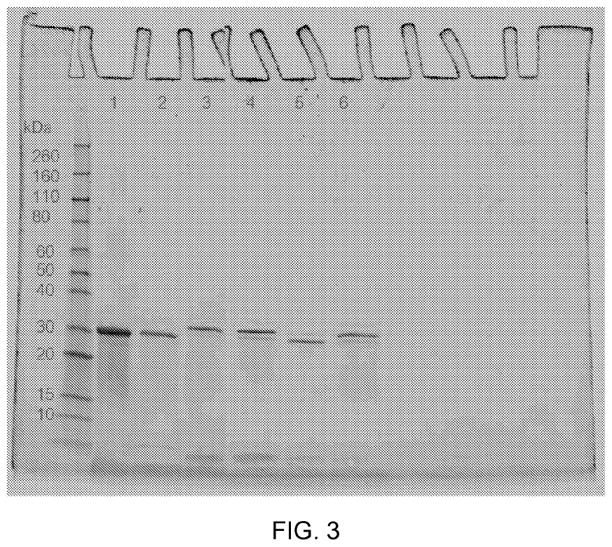

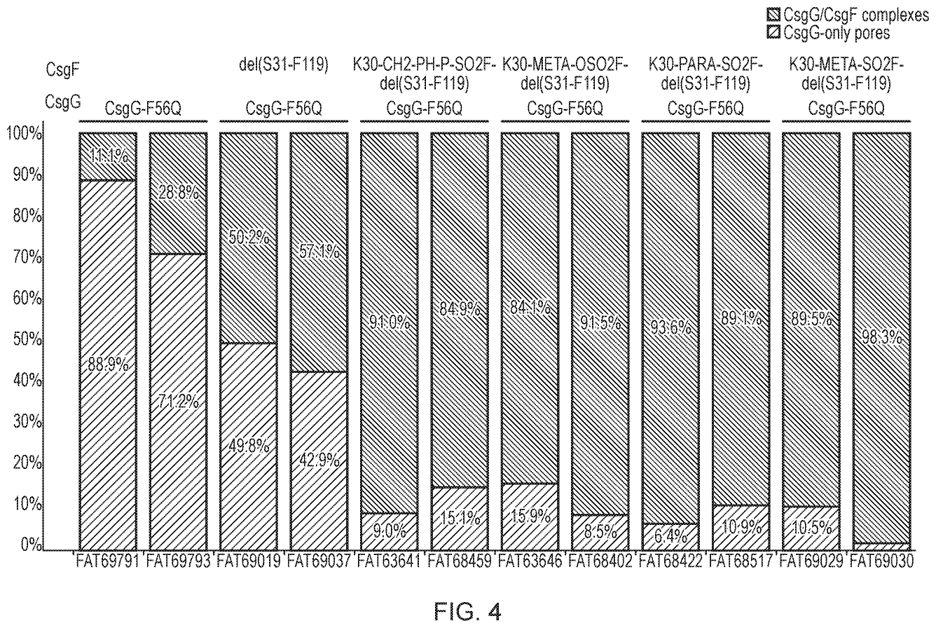

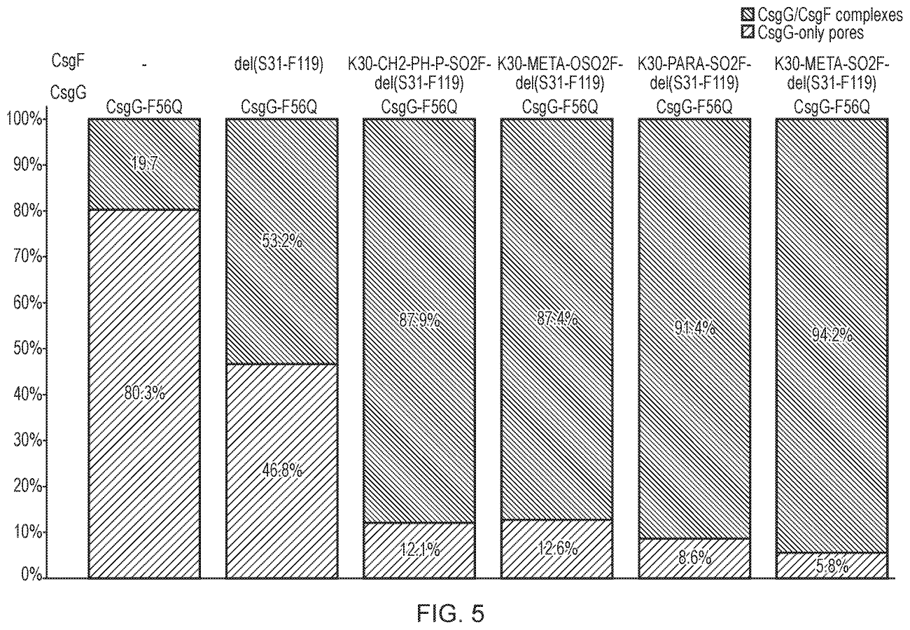

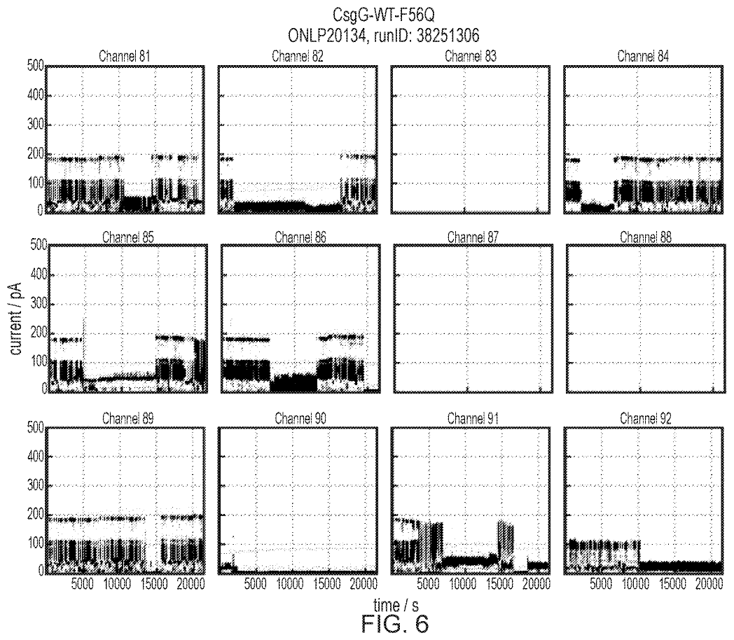

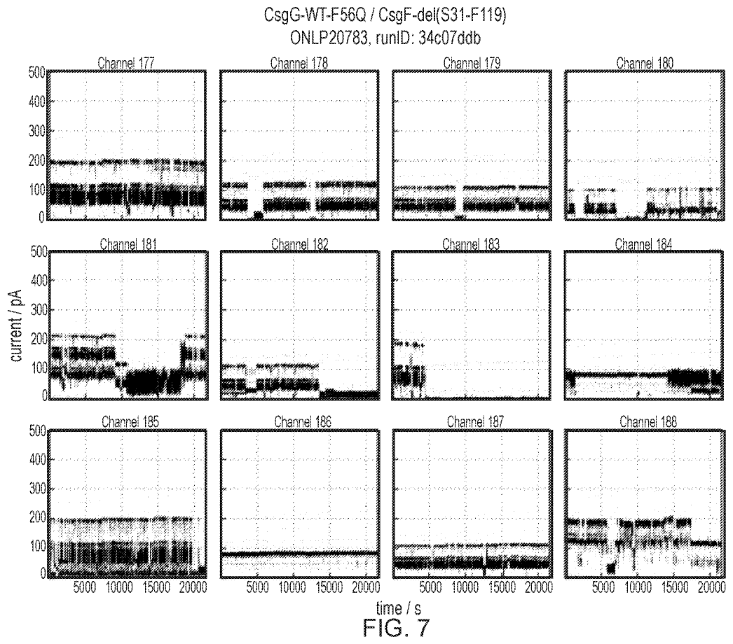

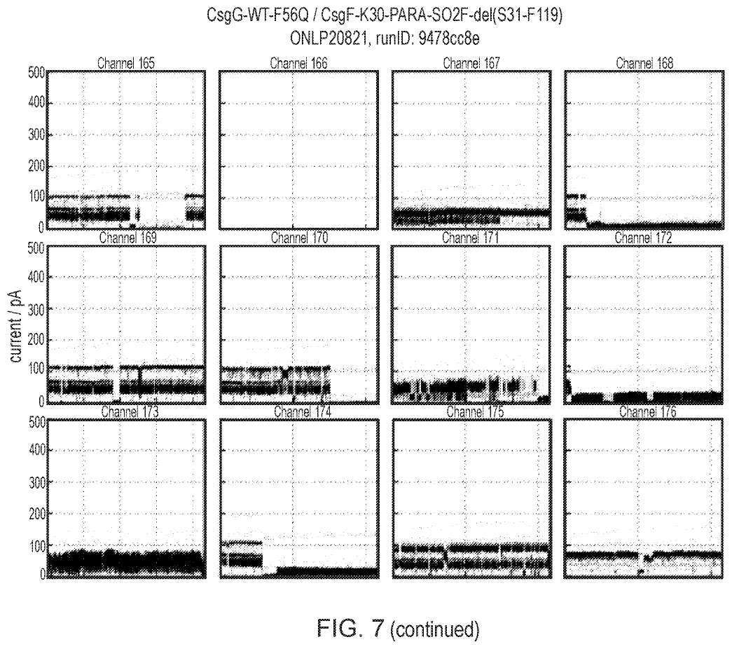

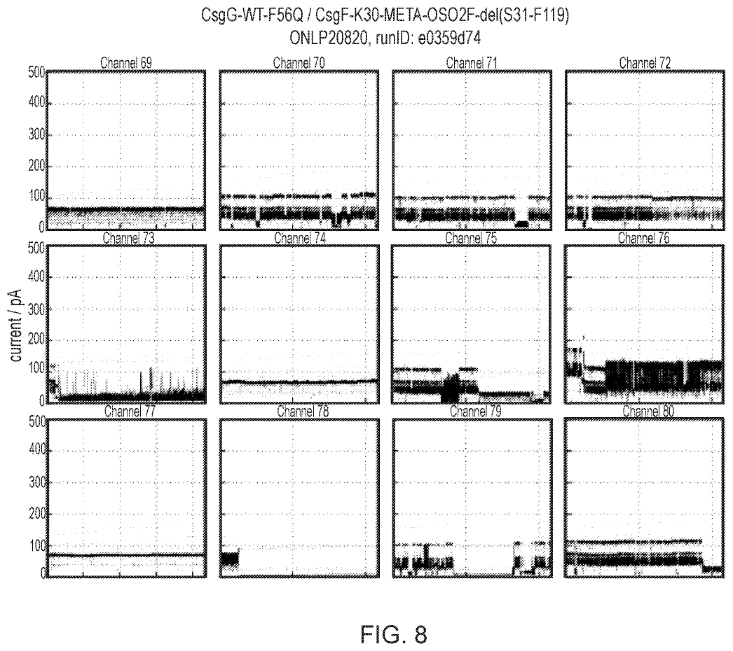

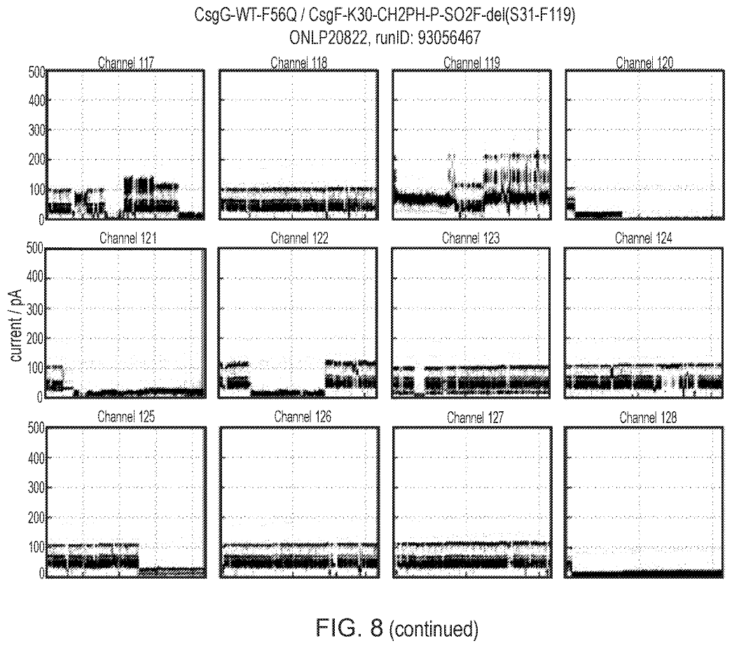

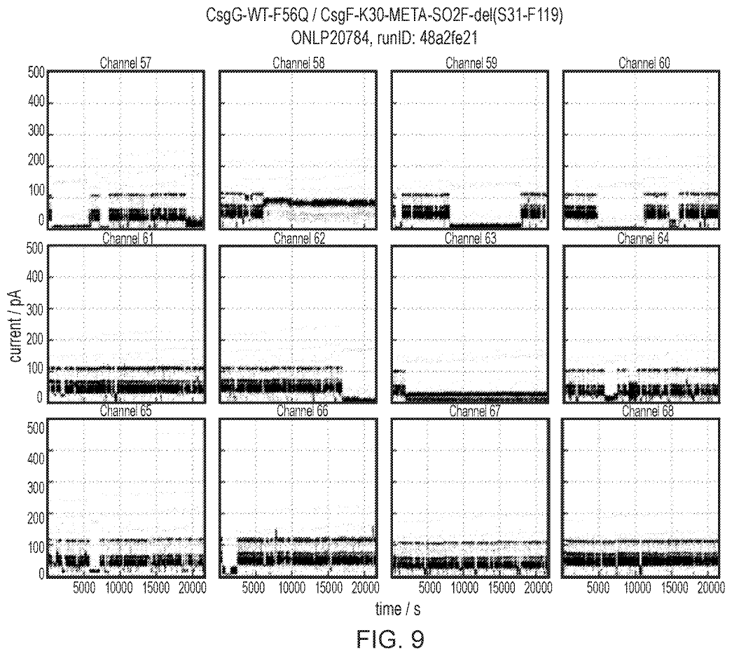

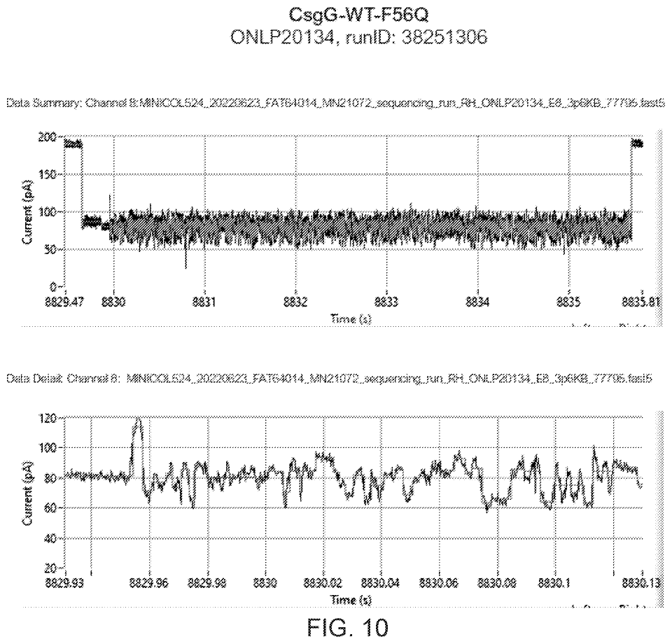

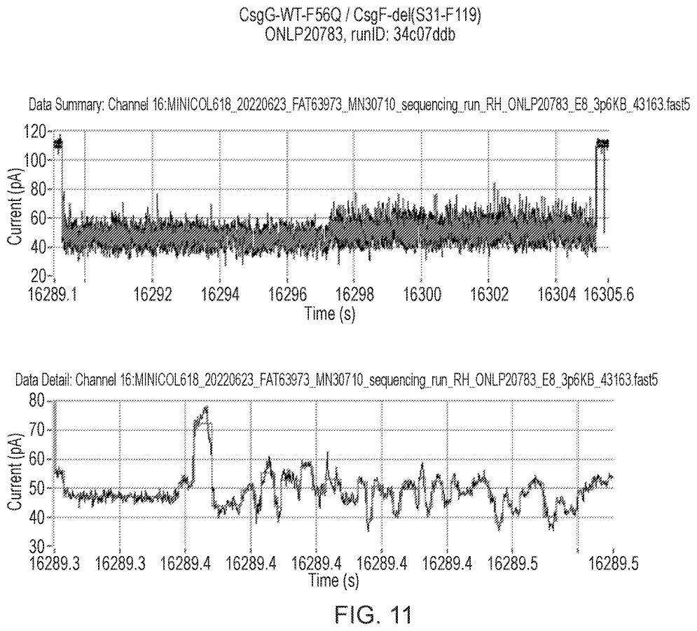

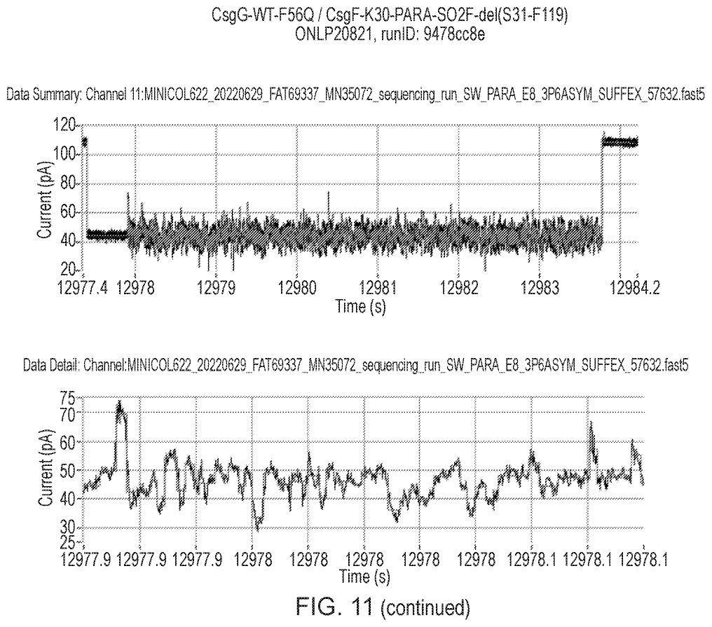

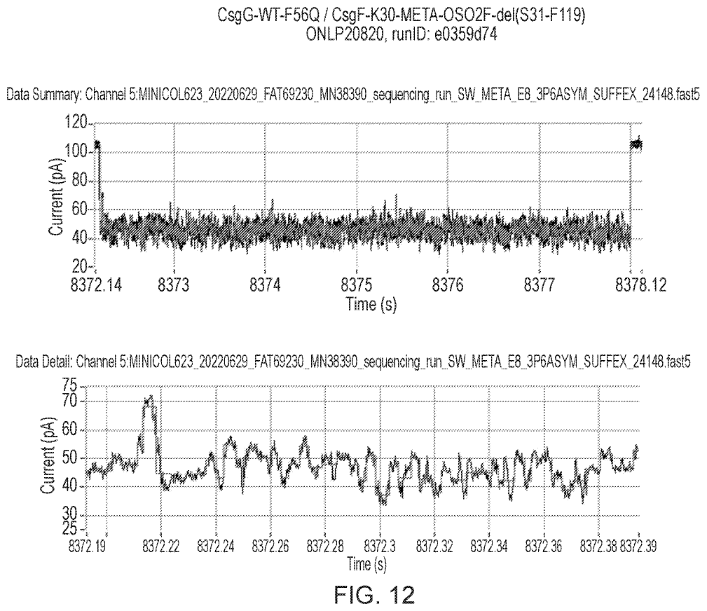

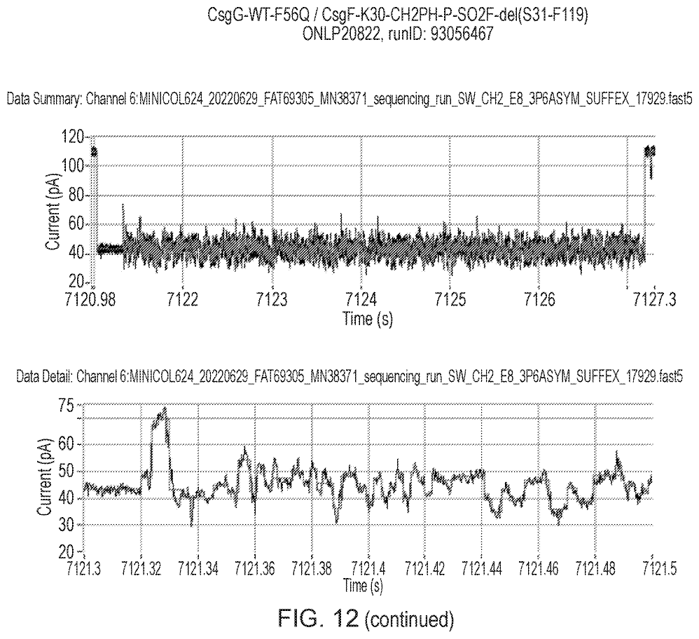

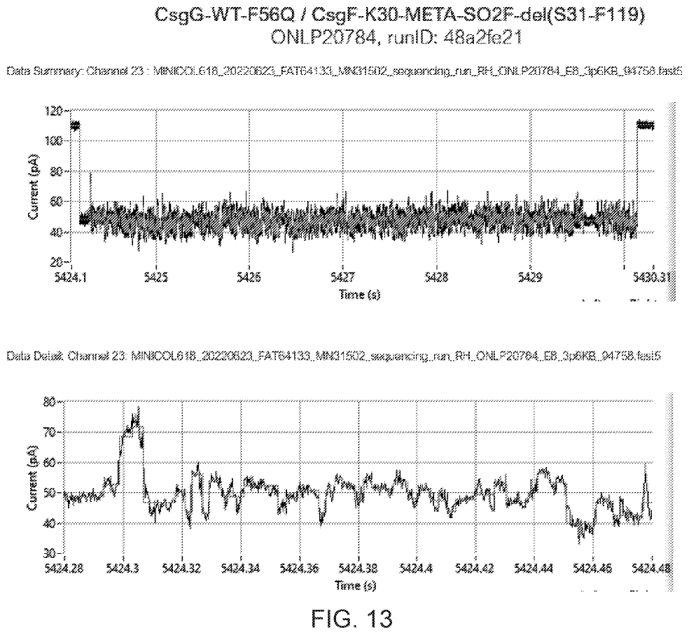

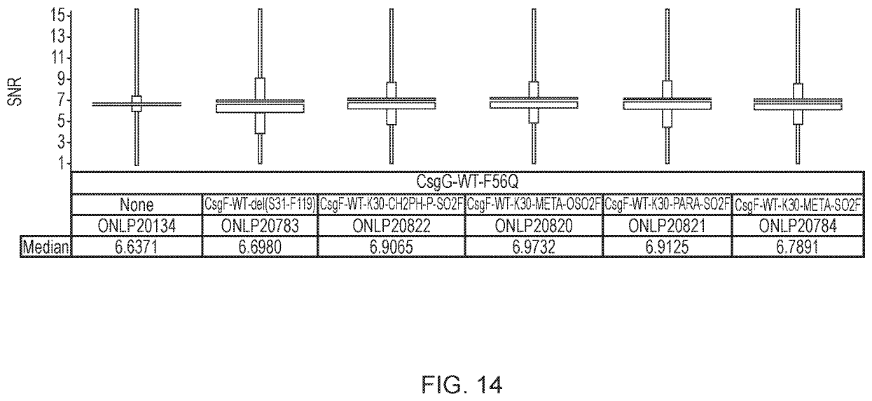

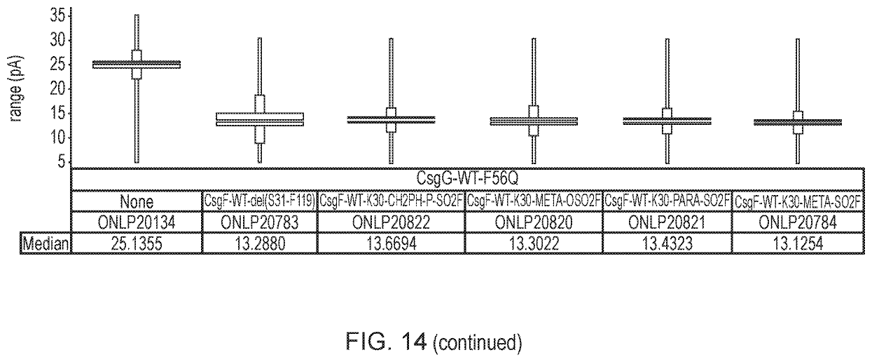

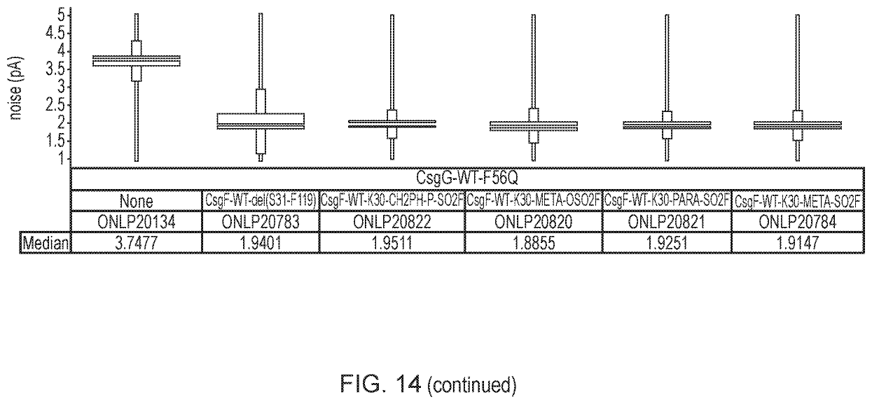

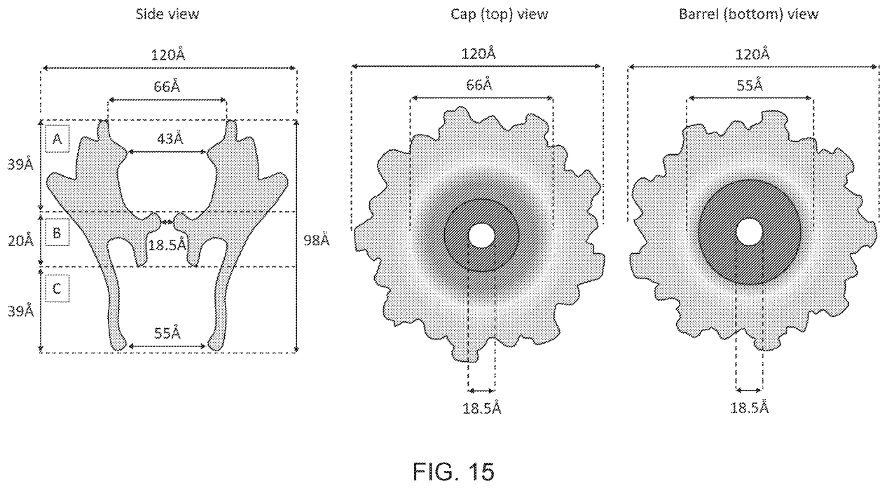

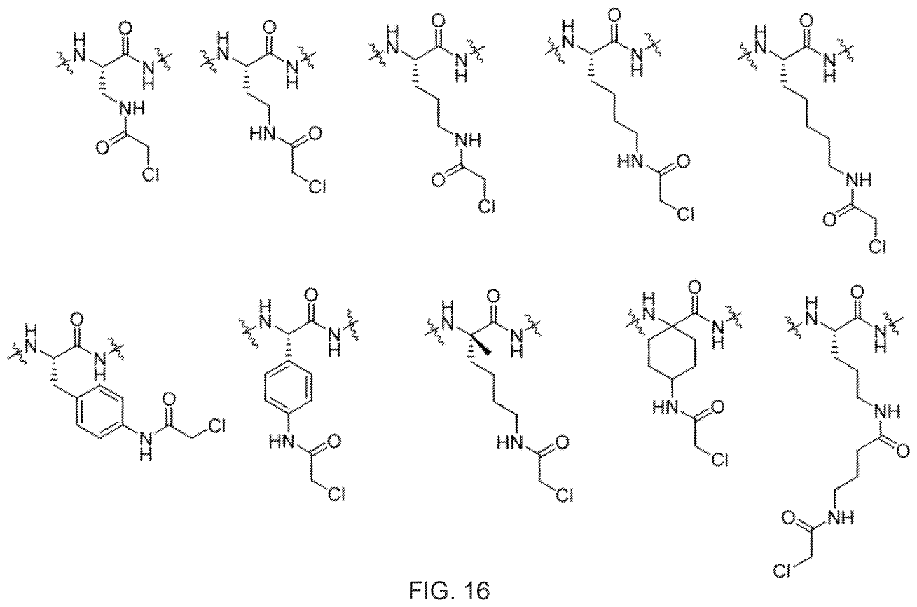

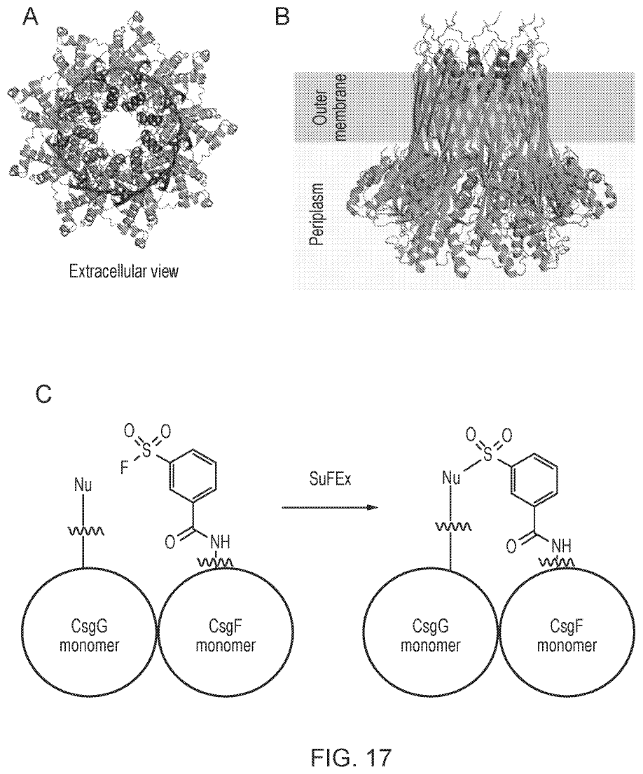

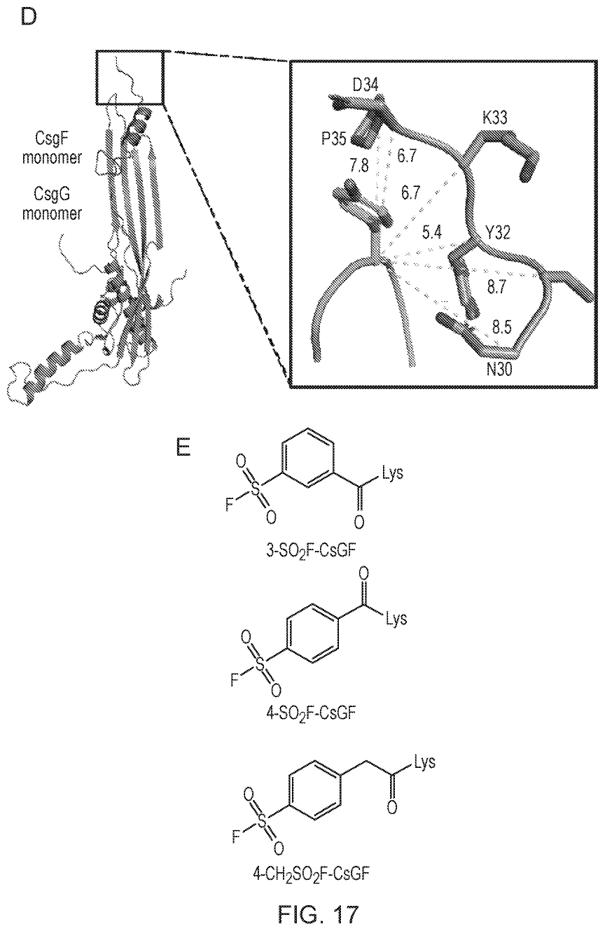

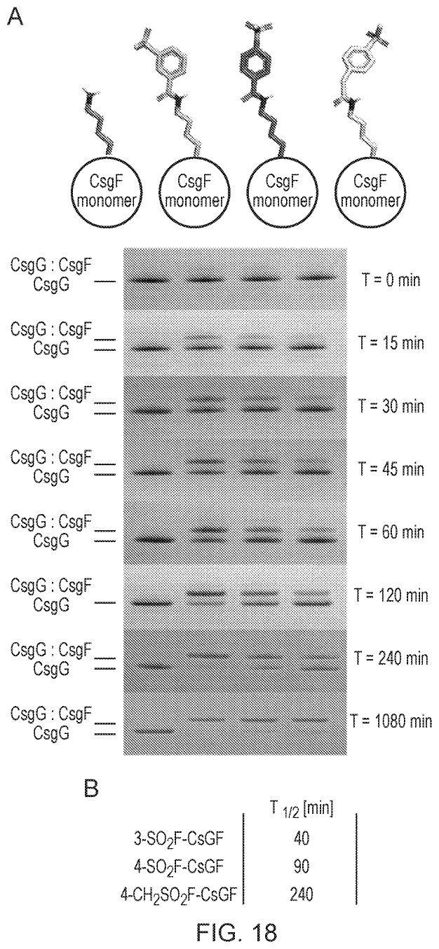

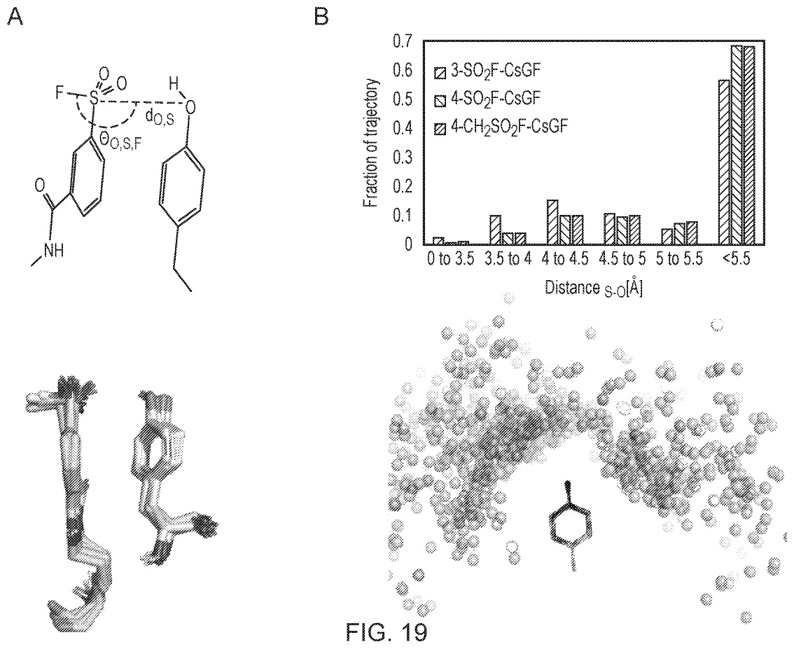

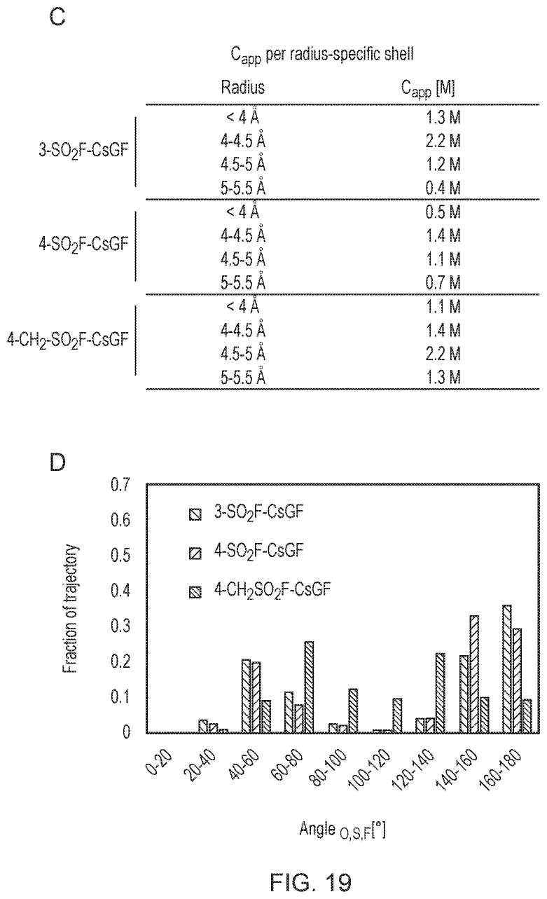

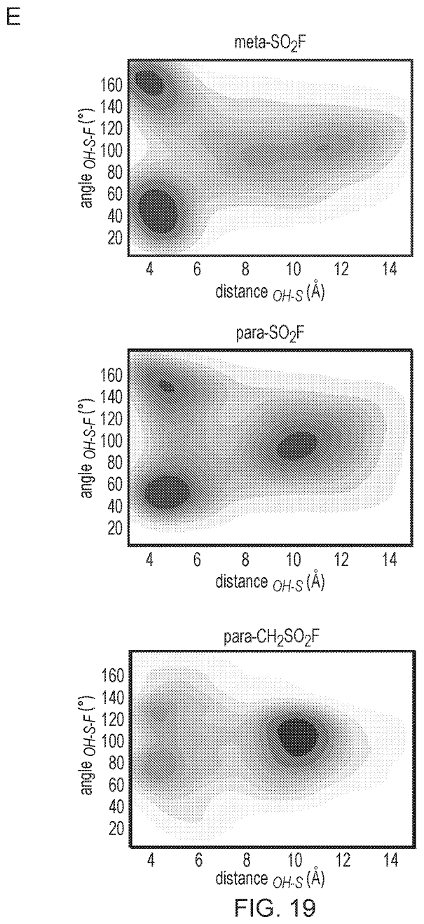

The linker may be any of the linkers discussed below with reference to the constructs of the invention. The linker preferably comprises or consists of (a) a sulfonyl fluoride, sulfonyl triazole, fluorosulfate, or fluoroacetamide group and (b) a linear carbon chain of 2, 3, 4, 5, 6 or more carbon atoms and/or saturated or unsaturated cyclic groups containing 3, 5 or 6 carbon atoms. Any linker may be used, including the ones used in the Examples (see FIGS. 1 and 2).

The linker is preferably a sulfonyl fluoride-containing linker. The linker is preferably CH₂PH—P—SO₂F, PARA-SO₂F, META-SO₂F, 4-(2-Aminoethyl)benzene-1-sulfonyl fluoride, ethylenesulfonyl fluoride, or 2-[(prop-2-yn-1-yl)oxy]ethane-1-sulfonyl fluoride.

The linker is preferably a sulfonyl triazole-containing linker. The linker is preferably substituted 4-(3-phenyl-1H-1,2,4-triazole-1-sulfonyl)benzoic acid, preferably H in FIG. 2 wherein X=H, OMe, CN, Br, Ph, or CF3, substituted 3-(3-phenyl-1H-1,2,4-triazole-1-sulfonyl)benzoic acid, 4-(1H-1,2,4-triazole-1-sulfonyl)benzoic acid, 4-[3-(pyridin-3-yl)-1H-1,2,4-triazole-1-sulfonyl]benzoic acid, or 3-[3-(pyridin-3-yl)-1H-1,2,4-triazole-1-sulfonyl]benzoic acid.

The linker is preferably a fluorosulfate-containing linker. The linker is preferably META-OSO₂F.

The linker is preferably a fluoroacetamide-containing linker. The linker is preferably 4-(2-fluoroacetamido)benzoic acid, or 3-(2-fluoroacetamido)benzoic acid.

The linker may be a residue in the CsgF peptide and/or the CsgG pore monomer modified to include a sulfonyl fluoride, sulfonyl triazole, fluorosulfate, or fluoroacetamide group. The CsgF peptide is preferably covalently attached to the CsgG pore monomer by the linker.

The reactive group in the linker may react with a residue in the CsgF peptide and/or in the CsgG pore monomer to attach the two together, preferably covalently attach the two together. The linker may comprise two reactive groups, such as two sulfonyl fluorides groups. One may react with the CsgF peptide and the other may react with the CsgG pore monomer. The reactive group in the linker preferably reacts with a serine, lysine, threonine, tyrosine, histidine, or cysteine residue in the CsgF peptide and/or in the CsgG pore monomer. These residues may be native residues in the CsgF peptide and/or the CsgG pore monomer. The residues may be introduced into the CsgF peptide and/or the CsgG pore monomer, preferably by substitution or addition.

SEQ ID NO: 6 shows the amino acid sequence of wild-type E. coli CsgF as a mature protein. The N terminus or the residue at any one of positions 1 to 40, such as position 1, 2, 3, 4, 5, 6, 7, 8, 9, 10, 11, 12, 13, 14, 15, 16, 17, 18, 19, 20, 21, 22 23, 24, 25, 26, 27, 28, 29, 30, 31, 32, 33, 34, 35, 36, 37, 38, 39, or 40, in the CsgF peptide is preferably attached to the CsgG pore monomer by the linker. The residue at any one of positions 23 to 40, such as position 23, 24, 25, 26, 27, 28, 29, 30, 31, 32, 33, 34, 35, 36, 37, 38, 39, or 40, in the CsgF peptide is preferably attached to the CsgG pore monomer by the linker. The residue at any one of positions 29 to 35, such as position 29, 30, 31, 32, 33, 34, or 35, in the CsgF peptide is preferably attached to the CsgG pore monomer by the linker.

The N terminus or the residue in the CsgF peptide corresponding to any of positions 1 to 40, such as position 1, 2, 3, 4, 5, 6, 7, 8, 9, 10, 11, 12, 13, 14, 15, 16, 17, 18, 19, 20, 21, 22 23, 24, 25, 26, 27, 28, 29, 30, 31, 32, 33, 34, 35, 36, 37, 38, 39, or 40, in the CsgF peptide is preferably attached to the CsgG pore monomer by the linker. The N terminus or the residue in the CsgF peptide corresponding to any of positions 1 to 40, such as position 1, 2, 3, 4, 5, 6, 7, 8, 9, 10, 11, 12, 13, 14, 15, 16, 17, 18, 19, 20, 21, 22 23, 24, 25, 26, 27, 28, 29, 30, 31, 32, 33, 34, 35, 36, 37, 38, 39, or 40, in SEQ ID NO:6 is preferably attached to the CsgG pore monomer by the linker. The residue in the CsgF peptide corresponding to any one of positions 23 to 40, such as corresponding to position 23, 24, 25, 26, 27, 28, 29, 30, 31, 32, 33, 34, 35, 36, 37, 38, 39, or 40, in SEQ ID NO: 6 is preferably attached to the CsgG pore monomer by the linker. The residue corresponding to any of positions 29 to 35, such as corresponding to position 29, 30, 31, 32, 33, 34, or 35, in SEQ ID NO: 6 is preferably attached to the CsgG pore monomer by the linker.

The residue at position 30 of the CsgF peptide or the residue in the CsgF peptide at the position corresponding to position 30 in SEQ ID NO: 6 is more preferably attached to the CsgG pore monomer by the linker.

The table below shows additional positions in the CsgF peptide that are preferably attached to the CsgG pore monomer by the linker. The positions in the left-hand column may be positions in the CsgF peptide or positions in SEQ ID NO: 6 to which the preferred positions correspond. The right-hand column shows preferred positions in the CsgG pore monomer to which the positions in the left-hand column are preferably attached by the linker. The CsgF peptide may be attached to any of the positions in the right-hand column by the linker.

| CsgF Peptide | |
|---|---|
| Residue | CsgG Monomer Residue |
| Gly1 | Lys49, Pro50, Ser132, Val134, Ser136, Gln151, Gln153, Ile181, Ser183, |
| | Glu185, Thr207, Asn209, Pro211, Val212 |
| Thr2 | Lys49, Pro50, Tyr51, Pro52, Ala53, Ala59, Ser132, Val134, Gln153, |
| | Ser183, Thr207, Asn209, Pro211, Val212 |
| Met3 | Pro50, Tyr51, Pro52, Ala53, Ser132, Val134, Ser136, Gln151, Gln153, |
| | Ser183, Glu185, Thr207, Asn209 |
| Thr4 | Pro52, Ala53, Ser132, Val134, Gln153, Ser136, Ser183, Glu185, Thr207, |
| | Asn209 |
| Phe5 | Val134, Ser136, Gly138, Gln151, Gln153, Glu185, Gln187, Gly205, |
| | Thr207 |
| Gln6 | Ser136, Gly138, Gln151, Gln153, Gln187, Gly205, Thr207 |
| Phe7 | Gly138, Gly140, Asp149, Gln151, Gln187, Gly189, Glu203, Gly205 |
| Arg8 | Gln187, Gly189, Glu203, Gly205 |
| Asn9 | Gly140, Gly189, Phe191, Glu201, Glu203 |
| Asn11 | Gly140, Arg142, Gly147, Asp149, Gln187, Gly189, Phe151, Glu201, |
| | Glu203 |
| Phe12 | Gly138, Gly140, Arg142, Gly147, Asp149, Gln151, Gln187, Gly189, |
| | Glu203 |
| Leu22 | Gly145, Phe191 |
| Leu23 | Phe191, Phe193, Leu199 |
| Ser25 | Phe144, Gly145, Phe191, Gln197, Arg198, Leu199 |
| Ala26 | Phe144, Gly145, Phe191, Phe193, Arg198, Leu199, |
| Gln29 | Phe144, Gly145, Phe191, Phe193, Asp195, Tyr196, Gln197, Arg198, |
| | Leu199 |
| Asn30 | Phe144, Gly145, Phe193, Asp195, Tyr196, Gln197, Arg198, Leu199 |
| Ser31 | Phe193, Asp195, Tyr196, Gln197, Arg198 |
| Tyr32 | Phe193, Asp195, Tyr196, Gln197, Arg198 |
| Lys33 | Asp195, Tyr196, Gln197 |
| Asp34 | Asp195, Tyr196, Gln197 |
| Pro35 | Asp195, Tyr196 |

SEQ ID NO: 3 shows the amino acid sequence of wild-type E. coli CsgG as a mature protein. The CsgF peptide is preferably attached to a residue in the loop forming regions of the CsgG pore monomer. The loop forming regions correspond to positions 142-146 and 190-200 in SEQ ID NO: 3. The CsgF peptide is preferably attached to a residue corresponding to positions 142-146 and 190-200 in SEQ ID NO: 3. The residue preferably corresponds to position 142, 143, 144, 145, 146, 190, 191, 192, 193, 194, 195, 196, 197, 198, 199 or 200 in SEQ ID NO: 3. Additional preferred positions in SEQ ID NO: 3 are shown in the right-hand column of the table above.

The CsgF peptide is preferably attached to a serine, lysine, threonine, tyrosine, histidine, or cysteine residue in the CsgG pore monomer by the linker. The sulfonyl fluoride group is capable of reacting with any of these residues. The sulfonyl fluoride group in the linker preferably reacts with a serine, lysine, threonine, tyrosine, histidine, or cysteine residue in the CsgG pore monomer. These residues may be native residues in CsgG pore monomer. The residues may be introduced into the CsgG pore monomer, preferably by substitution or addition.

In another embodiment, the CsgF peptide is attached to a residue in the CsgG pore monomer corresponding to any one of positions 143, 146, 190, 192, 193, 194, 195, 196, 197, 198, 199 or 200 in SEQ ID NO: 3. The CsgF peptide is more preferably attached to a residue in the CsgG pore monomer corresponding to position 196 in SEQ ID NO: 3. The CsgF peptide may be attached, preferably covalently attached, to any one of these positions using a linker. The linker may be any of those described above and below. The linker may comprise a sulfonyl fluoride group, a sulfonyl triazole group, a fluorosulfate group, or a fluoroacetamide group.

The residue corresponding to any one of positions 143, 146, 190, 192, 193, 194, 195, 196, 197, 198, 199 or 200 in SEQ ID NO: 3 is preferably a serine, lysine, threonine, tyrosine, histidine, cysteine, or threonine residue. Any of these residues may be native residues. Any of these residues may be introduced into the CsgG pore monomer, preferably by substitution or addition.

The residue corresponding to any one of positions 143, 146, 190, 192, 193, 194, 195, 196, 197, 198, 199 or 200 in SEQ ID NO: 3 is preferably modified with a reactive group. Any of the reactive groups described above or below may be used. For example, the residue is preferably a derivative of diaminopropionic acid, diaminobutyric acid, ornithine, lysine (Lys), homo-Lys, p-amino phenylalanine, p-amino Phenylglycine, alpha-methyllysine or 1,4-diaminocyclohexane-1-carboxylic acid in which the sidechain amino group is covalently attached to a reactive group (e.g., an alpha-chloro acetamide) with or without linker. Examples are shown in FIG. 16.

The CsgF peptide is preferably attached to the CsgG pore monomer using any reactive group capable of reacting with the residue corresponding to any one of positions 143, 146, 190, 192, 193, 194, 195, 196, 197, 198, 199 or 200 in SEQ ID NO: 3, a residue introduced, preferably by substitution or addition, at any one of these positions, or a reactive group introduced at any one of these positions.

The reactive group is preferably an amine-reactive group, such as a thioester, NHS-ester, pentafluorophenyl ester, benzylic halide, sulfonyl fluoride, fluorosulfate, or sulfonyl triazole. Suitable sulfonyl-triazole groups are discussed above and shown in FIG. 2 (see H-L). The reactive group is preferably an amine-reactive group, such as a thioester, NHS-ester, pentafluorophenyl ester, benzylic halide, benzylic fluoride, sulfonyl halide, sulfonyl fluoride, halosulfate, fluorosulfate, sulfonyl triazole, or boronic acid. Suitable sulfonyl-triazole groups are discussed above and shown in FIG. 2 (see H-L).

The reactive group is preferably an oxygen-reactive group, such as an alkyl halide, sulfonyl fluoride, fluorosulfate, or sulfonyl triazoles. The alkyl halide is preferably a chloromethyl ketone. The reactive group is preferably an oxygen-reactive group, such as an alkyl halide, alkyl fluoride, sulfonyl halide, sulfonyl fluoride, halosulfate, fluorosulfate, or sulfonyl triazoles. The alkyl halide is preferably a chloromethyl ketone.

The reactive group is preferably a fluoroacetamide group. Suitable fluoroacetamide groups are discussed above and are shown in FIG. 2 (see M and N).

Additional reactive groups are described in Nature Chemistry, 2021, 13, 1081-1092, Cell Chemical Biology, 2020, 27, 970-985, J. Am. Chem. Soc. 2019, 141, 7, 2782-2799, and Current Opinion in Chemical Biology, 2015, 18-26 (each incorporated herein by reference in their entirety).

Any residue in the CsgF peptide may be attached to any one of the residues at positions corresponding to 143, 146, 190, 192, 193, 194, 195, 196, 197, 198, 199 or 200 in SEQ ID NO: 3. The residue in CsgF is preferably any of those discussed above with reference to the sulfonyl fluoride, sulfonyl triazole, fluorosulfate, and fluoroacetamide embodiments of the invention.

The distance between the CsgG pore monomer and the CsgF peptide in the pore monomer conjugate and/or the length of the linker is preferably less than about 3.00 nm, such as less than about 2.90 nm, less than about 2.80 nm, less than about 2.70 nm, less than about 2.60 nm, less than about 2.50 nm, less than about 2.40 nm, less than about 2.30 nm, less than about 2.20 nm, less than about 2.10, less than about 2.00 nm, less than about 1.90 nm, less than about 1.80 nm, less than about 1.70 nm, less than about 1.60 nm, less than about 1.50 nm, less than about 1.40 nm, less than about 1.30 nm, less than about 1.20 nm, less than about 1.10 nm, less than about 1.00 nm, less than about 0.90 nm, less than about 0.80 nm, less than about 0.70 nm, less than about 0.60 nm, less than about 0.50 nm, or less than about 0.40 nm.

The distance between the CsgG pore monomer and the CsgF peptide in the pore monomer conjugate and/or the length of the linker is preferably from about 0.40 nm to about 3.00 nm, such as about 0.45 nm to about 2.80 nm, from about 0.50 nm to about 2.50 nm, from about 0.55 nm to about 2.20 nm, from about 0.60 nm to about 2.00 nm, from about 0.65 nm to about 1.50 nm, from about 0.70 nm to about 1.40 nm, from about 0.75 nm to about 1.30 nm, from about 0.80 nm to about 1.20 nm, from about 0.85 nm to about 1.10 nm and from about 0.90 nm to about 1.00 nm. The distance between the CsgG pore monomer and the CsgF peptide in the pore monomer conjugate and/or the length of the linker is preferably from about 0.50 nm to about 1.50 nm. The distance between the CsgG pore monomer and the CsgF peptide in the pore monomer conjugate and/or the length of the linker is preferably from about 0.60 nm to about 1.20 nm.

The pore monomer conjugates of the invention are capable of forming a pore or a pore complex. This can be measured using routine methods, including any of those described in WO 2016/034591, WO 2017/149316, WO 2017/149317, WO 2017/149318, WO 2018/211241, and WO 2019/002893 (all incorporated by reference herein in their entirety) and in the Examples.

### CsgG Pore Monomer

A CsgG pore monomer is a monomer that is capable of forming a CsgG pore. Such monomers are known in the art, especially from WO 2019/002893 (incorporated by reference herein in its entirety). The CsgG pore preferably comprises one or more of (a) a cap region, (b) a constriction region, and (c) a transmembrane beta barrel region, such as (a), (b), (c), (a) and (b), (a) and (c), (b) and (c), or (a), (b) and (c). The CsgG pore monomer preferably comprises one or more of (a) a cap forming region, (b) a constriction forming region, and (c) a transmembrane beta barrel forming region, such as (a), (b), (c), (a) and (b), (a) and (c), (b) and (c), or (a), (b) and (c). The residues of SEQ ID NO: 3 which form these regions are defined below. The CsgG pore formed by the monomer may have any structure but preferably has or comprises the structure of the wild-type CsgG pore (FIG. 15). The protein structure of CsgG defines a channel or hole that allows the translocation of molecules and ions from one side of the membrane to the other.

The “constriction”, “orifice”, “constriction region”, “channel constriction”, or “constriction site”, as used interchangeably herein, refers to an aperture defined by a luminal surface of a pore or pore complex, which acts to allow the passage of ions and target molecules (e.g., but not limited to polynucleotides or individual nucleotides) but not other non-target molecules through the pore or pore complex channel. The constriction(s) are typically the narrowest aperture(s) within a pore or pore complex or within the channel defined by the pore or pore complex. The constriction(s) may serve to limit the passage of molecules through the pore. The size of the constriction is typically a key factor in determining suitability of a pore or pore complex for analyte characterisation. If the constriction is too small, the molecule to be characterised will not be able to pass through. However, to achieve a maximal effect on ion flow through the channel, the constriction should not be too large. For example, the constriction should not be wider than the solvent-accessible transverse diameter of a target analyte. Ideally, any constriction should be as close as possible in diameter to the transverse diameter of the analyte passing through. The CsgF peptide and the CsgG pore monomer typically each provide at least one constriction such that the pore complex of the invention comprises two or more constrictions.

The CsgG pore may be any size but preferably has the dimensions of the wild-type CsgG pore (FIG. 15). The CsgG pore preferably has an external diameter of from about 100 to about 150 Å at its widest point, such as from about 110 to about 140 Å or from about 115 to about 125 Å at its widest point. The CsgG pore preferably has an external diameter of about 120 Å at its widest point. The CsgG pore preferably has a total length of from about 80 to about 120 Å, such as from about 90 to about 110 Å or from about 95 to about 105 Å. The CsgG pore preferably has a total length of about 98 Å. References to “total length” and “length” relate to the length of the pore or pore region when viewed from the side (see, e.g., the side view in FIG. 15).

The cap region preferably has a length of from about 20 to about 60 Å, such as from about 30 to about 50 Å or from about 35 to about 45 Å. The cap region preferably has a length of about 39 A. The channel defined by the cap region preferably has an opening of from about 45 to about 85 Å in diameter, such as from about 55 to about 75 Å or from about 60 to about 70 Å in diameter. The channel defined by the cap region preferably has an opening of about 66 Å in diameter. The channel defined by the cap region is preferably from about 30 to about 70 Å in diameter at its narrowest point, such as from about 35 to about 60 Å or from about 40 to about 50 Å in diameter at its narrowest point. The channel defined by the cap region is preferably about 43 Å in diameter at its narrowest point.

The constriction region preferably has a length of from about 5 to about 40 Å, such as from about 10 to about 30 Å or from about 15 to about 25 Å. The constriction region preferably has a length of about 20 Å. The channel defined by the constriction region is preferably from about 2 to about 40 Å in diameter at its narrowest point, such as from about 5 to about 35 Å, from about 8 to about 25 Å or from about 10 to about 20 Å in diameter at its narrowest point. The channel defined by the constriction region is preferably about 9 Å or 12 Å in diameter. The channel defined by the constriction region is preferably about 18.5 Å in diameter. The constriction is preferably from about 2 to about 40 Å in diameter, such as from about 5 to about 35 Å, from about 8 to about 25 Å or from about 10 to about 20 Å in diameter. The constriction is preferably about 9 Å or 12 Å in diameter. The constriction is preferably about 12 Å in diameter.

The transmembrane beta barrel region preferably has a length of from about 20 to about 60 Å, such as from about 30 to about 50 Å or from about 35 to about 45 Å. The transmembrane beta barrel preferably has a length of about 39 Å. The channel defined by the transmembrane beta barrel region is preferably from about 35 to about 75 Å in diameter at its narrowest point, such as from about 45 to about 65 Å or from about 50 to about 60 Å in diameter at its narrowest point. The channel defined by the transmembrane beta barrel region is preferably about 55 Å in diameter at its narrowest point.

All of the measurements above are based on measuring from backbone to backbone of the amino acids forming the different regions (as shown in FIG. 15).

SEQ ID NO: 3 shows the sequence of wild-type E. coli CsgG as a mature protein. Residues 1 to 41, 64 to 131, 156 to 180 and 212 to 262 of SEQ ID NO: 3 form the cap region. Residues 42 to 63 of SEQ ID NO: 3 form the constriction region. Residues 132 to 155 and 181 to 211 of SEQ ID NO: 3 form the transmembrane beta barrel region.

The CsgG pore monomer is preferably a variant of SEQ ID NO: 3. The variant CsgG monomer may also be referred to as a modified CsgG pore monomer or a mutant CsgG pore monomer. The modifications, or mutations, in the variant include but are not limited to any one or more of the modifications disclosed herein, or combinations of said modifications. The CsgG pore monomer may be a CsgG homologue monomer. A CsgG homologue monomer is a polypeptide that has at least 40%, 50%, 60%, 70%, 80%, 85%, 90%, 95% or 99% complete sequence identity to wild-type E. coli CsgG as shown in SEQ ID NO: 3. A CsgG homologue is also referred to as a polypeptide that contains the PFAM domain PF03783, which is characteristic for CsgG-like proteins. A list of presently known CsgG homologues and CsgG architectures can be found at http://pfam.xfam.org//family/PF03783.

Over the entire length of the amino acid sequence of SEQ ID NO: 3, a variant will preferably be at least 40% homologous to that sequence based on amino acid identity. More preferably, the variant may be at least 45%, at least 50%, at least 55%, at least 60%, at least 65%, at least 70%, at least 75%, at least 80%, at least 85%, at least 90% and more preferably at least 95%, 97% or 99% homologous based on amino acid identity to the amino acid sequence of SEQ ID NO: 3 over the entire sequence. Over the entire length of the amino acid sequence of SEQ ID NO: 3, a variant will preferably be at least 40% identical to that sequence. More preferably, the variant may be at least 45%, at least 50%, at least 55%, at least 60%, at least 65%, at least 70%, at least 75%, at least 80%, at least 85%, at least 90% and more preferably at least 95%, 97% or 99% identical to SEQ ID NO: 3 over the entire sequence.

Sequence identity can also relate to a fragment or portion of the CsgG pore monomer. Hence, a sequence may have less than 40% overall sequence homology/identity with SEQ ID NO: 3, but the sequence of a particular region, domain or subunit could share at least 80%, 90%, or as much as 99% sequence homology/identity with the corresponding region of SEQ ID NO: 3. There may be at least 80%, for example at least 85%, 90% or 95%, amino acid identity over a stretch of 100 or more, for example 125, 150, 175 or 200 or more, contiguous amino acids (“hard homology”). The CsgG pore monomer is preferably a variant of SEQ ID NO: 3 comprising a sequence that is at least 40% homologous to the cap region of SEQ ID NO: 3 (residues 1 to 41, 64 to 131, 156 to 180 and 212 to 262). More preferably, the variant may comprise a sequence that is at least 45%, at least 50%, at least 55%, at least 60%, at least 65%, at least 70%, at least 75%, at least 80%, at least 85%, at least 90% and more preferably at least 95%, 97% or 99% homologous based on amino acid identity to residues 1 to 41, 64 to 131, 156 to 180 and 212 to 262 of SEQ ID NO: 3. The variant preferably comprises a sequence that is at least 40% identical to residues 1 to 41, 64 to 131, 156 to 180 and 212 to 262 of SEQ ID NO: 3. More preferably, the variant may comprise a sequence that is at least 45%, at least 50%, at least 55%, at least 60%, at least 65%, at least 70%, at least 75%, at least 80%, at least 85%, at least 90% and more preferably at least 95%, 97% or 99% identical to residues of 1 to 41, 64 to 131, 156 to 180 and 212 to 262 of SEQ ID NO: 3. Homology and/or identity is typically measured over the entire length of the cap region.

The CsgG pore monomer is preferably a variant of SEQ ID NO: 3 comprising a sequence that is at least 40% homologous to the constriction region of SEQ ID NO: 3 (residues 42 to 63). More preferably, the variant may comprise a sequence that is at least 45%, at least 50%, at least 55%, at least 60%, at least 65%, at least 70%, at least 75%, at least 80%, at least 85%, at least 90% and more preferably at least 95%, 97% or 99% homologous based on amino acid identity to residues 42 to 63 of SEQ ID NO: 3. The variant preferably comprises a sequence that is at least 40% identical to residues 42 to 63 of SEQ ID NO: 3. More preferably, the variant may comprise a sequence that is at least 45%, at least 50%, at least 55%, at least 60%, at least 65%, at least 70%, at least 75%, at least 80%, at least 85%, at least 90% and more preferably at least 95%, 97% or 99% identical to residues of 42 to 63 of SEQ ID NO: 3. Homology and/or identity is typically measured over the entire length of the constriction region.

The CsgG pore monomer is preferably a variant of SEQ ID NO: 3 comprising a sequence that is at least 40% homologous to the transmembrane beta barrel region of SEQ ID NO: 3 (residues 132 to 155 and 181 to 211). More preferably, the variant may comprise a sequence that is at least 45%, at least 50%, at least 55%, at least 60%, at least 65%, at least 70%, at least 75%, at least 80%, at least 85%, at least 90% and more preferably at least 95%, 97% or 99% homologous based on amino acid identity to residues 132 to 155 and 181 to 211 of SEQ ID NO: 3. The variant preferably comprises a sequence that is at least 40% identical to residues 132 to 155 and 181 to 211 of SEQ ID NO: 3. More preferably, the variant may comprise a sequence that is at least 45%, at least 50%, at least 55%, at least 60%, at least 65%, at least 70%, at least 75%, at least 80%, at least 85%, at least 90% and more preferably at least 95%, 97% or 99% identical to residues of 132 to 155 and 181 to 211 of SEQ ID NO: 3. Homology and/or identity is typically measured over the entire length of the transmembrane beta barrel region.

CsgG pore monomers are highly conserved (as can be readily appreciated from FIGS. 45 to 47 of WO 2017/149317). Furthermore, from knowledge of the mutations in relation to SEQ ID NO: 3 it is possible to determine the equivalent positions for mutations of CsgG pore monomers other than that of SEQ ID NO: 3.

Thus, reference to a mutant CsgG pore monomer comprising a variant of the sequence as shown in SEQ ID NO: 3 and specific amino-acid mutations thereof as set out in the claims and elsewhere in the specification also encompasses a mutant CsgG pore monomer comprising a variant of any of the sequences shown in SEQ ID NOs: 68 to 88 of WO 2019/002893 (incorporated by reference herein in its entirety) and corresponding amino-acid mutations thereof. The CsgG pore monomer may also be any of the sequences shown in CN 113773373 A, CN 113896776 A, CN 113912683 A, and CN 113754743 A or a variant thereof. It will further be appreciated that the invention extends to other variant CsgG pore monomers not expressly identified in the specification that show highly conserved regions.

Standard methods in the art may be used to determine homology. For example, the UWGCG Package provides the BESTFIT program which can be used to calculate homology, for example used on its default settings (Devereux et al (1984) Nucleic Acids Research 12, p 387-395). The PILEUP and BLAST algorithms can be used to calculate homology or line up sequences (such as identifying equivalent residues or corresponding sequences (typically on their default settings)), for example as described in Altschul S. F. (1993) J Mol Evol 36:290-300; Altschul, S. F et al (1990) J Mol Biol 215:403-10. Software for performing BLAST analyses is publicly available through the National Center for Biotechnology Information (http://www.ncbi.nlm.nih.gov/).

SEQ ID NO: 3 is the wild-type CsgG pore monomer from Escherichia coli Str. K-12 substr. MC4100. A variant of SEQ ID NO: 3 may comprise any of the substitutions present in another CsgG homologue. Preferred CsgG homologues are shown in SEQ ID NOs: 68 to 88 of WO 2019/002893 (incorporated by reference herein in its entirety). The variant may comprise combinations of one or more of the substitutions present in SEQ ID NOs: 68 to 88 WO 2019/002893 (incorporated by reference herein in its entirety) compared with SEQ ID NO: 3, including one or more substitutions, one or more conservative mutations, one or more deletions or one or more insertion mutations, such as deletion or insertion of 1 to 10 amino acids, such as of 2 to 8 or 3 to 6 amino acids.

The CsgG pore monomer in the pore monomer conjugate of the invention typically retains the ability to form the same 3D structure as the wild-type CsgG pore monomer, such as the same 3D structure as a CsgG pore monomer having the sequence of SEQ ID NO: 3. The 3D structure of CsgG is known in the art and is disclosed, for example, in Goyal et al (2014) Nature 516(7530):250-3. Any number of mutations may be made in the wild-type CsgG sequence in addition to the mutations described herein provided that the CsgG pore monomer retains the improved properties imparted on it by the mutations of the present invention.

Typically, the CsgG pore monomer will retain the ability to form a structure comprising five alpha-helices and five beta-strands. Therefore, it is envisaged that further mutations may be made in any of these regions in any CsgG pore monomer without affecting the ability of the monomer to form a pore that can translocate polynucleotides. It is also expected that deletions of one or more amino acids can be made in any of the loop regions linking the alpha helices and beta-strands and/or in the N-terminal and/or C-terminal regions of the CsgG pore monomer without affecting the ability of the monomer to form a pore that can translocate polynucleotides.

Amino acid substitutions may be made to the amino acid sequence of SEQ ID NO: 3 in addition to those discussed above, for example up to 1, 2, 3, 4, 5, 10, 20 or 30 substitutions. Conservative substitutions replace amino acids with other amino acids of similar chemical structure, similar chemical properties, or similar side-chain volume. The amino acids introduced may have similar polarity, hydrophilicity, hydrophobicity, basicity, acidity, neutrality, or charge to the amino acids they replace. Alternatively, the conservative substitution may introduce another amino acid that is aromatic or aliphatic in the place of a pre-existing aromatic or aliphatic amino acid. Conservative amino acid changes are well-known in the art.

The CsgG pore monomer may be modified to introduce one or more cysteines, one or more hydrophobic amino acids, one or more charged amino acids, one or more non-native amino acids, one or more polar amino acids, or one or more photoreactive amino acids. Any number and combination of such introductions may be made. The introduction is preferably by substitution or addition.

One or more amino acid residues of the amino acid sequence of SEQ ID NO: 3 may additionally be deleted from the polypeptides described above. Up to 1, 2, 3, 4, 5, 10, 20 or 30 or more residues may be deleted.

Variants may include fragments of SEQ ID NO: 3. Such fragments retain pore forming activity. Fragments may be at least 50, at least 100, at least 150, at least 200 or at least 250 amino acids in length. Such fragments may be used to produce the pores. A fragment preferably comprises the transmembrane beta barrel region of SEQ ID NO: 3, namely residues 132 to 155 and 181 to 211, or a variant thereof as discussed above.

One or more amino acids may be alternatively or additionally added to the polypeptides described above. An extension may be provided at the amino terminal or carboxy terminal of the amino acid sequence of SEQ ID NO: 3 or polypeptide variant or fragment thereof. The extension may be quite short, for example from 1 to 10 amino acids in length. Alternatively, the extension may be longer, for example up to 50 or 100 amino acids. A carrier protein may be fused to an amino acid sequence according to the invention. Other fusion proteins are discussed in more detail below.

A variant of SEQ ID NO: 3 is a polypeptide that has an amino acid sequence which varies from that of SEQ ID NO: 3 and which retains its ability to form a pore. A variant typically contains the regions of SEQ ID NO: 3 that are responsible for pore formation. The pore forming ability of CsgG, which contains a □-barrel, is provided by □-strands in the transmembrane beta barrel region of each monomer. A variant of SEQ ID NO: 3 typically comprises the region in SEQ ID NO: 3 that forms □-strands, namely residues 132 to 155 and 181 to 211, or a variant thereof as discussed above. One or more modifications can be made to the region of SEQ ID NO: 3 that form □-strands as long as the resulting variant retains its ability to form a pore.

The one or more modifications in the CsgG pore monomer preferably improve the ability of a pore complex comprising the pore monomer to characterise an analyte. For example, modifications/mutations/substitutions are contemplated to alter the number, size, shape, placement, or orientation of the constriction within a channel from the pore monomer conjugate of the invention. The CsgG pore monomer or the variant of SEQ ID NO: 3 may have any of the particular modifications or substitutions disclosed in WO 2016/034591, WO 2017/149316, WO 2017/149317, WO 2017/149318, WO 2018/211241, and WO 2019/002893 (all incorporated by reference herein in their entirety).

Preferred modifications or substitutions in SEQ ID NO: 3 include, but are not limited to, one or more of, such as 2 or more, 3 or more, 4 or more, 5 or more, 6 or more, 7 or more or all of:
(a) a substitution at position Y51, such as Y51I, Y51L, Y51A, Y51V, Y51T, Y51S, Y51Q or Y51N;
(b) a substitution at position N55, such as N55I, N55L, N55A, N55V, N55T, N55S or N55Q;
(c) a substitution at position F56, such as F56I, F56L, F56A, F56V, F56T, F56S, F56Q or F56N;
(d) a substitution at position L90, such as L90N, L90D, L90E, L90R or L90K;
(e) a substitution at position N91, such as N91D, N91E, N91R or N91K;
(f) a substitution at position K94, such as K94R, K94F, K94Y, K94Q, K94W, K94L, K94S or K94N;
(g) a substitution at position R192, such as R192Q, R192F, R192S R192D, or R192T; and
(i) a substitution at position C215, such as C215T, C215S, C215I, C215L, C215A, C215V, or C215G.

The variant of SEQ ID NO: 3 may further comprise a deletion of one or more positions, such as a deletion of T104-N109, a deletion of F193-L199 or a deletion of F195-L199.

Any number of the CsgG pore monomers in the pore or pore complex of the invention, such as 6, 7, 8, 9 or 10, may be a variant of SEQ ID NO: 3. All six to ten monomers in the pore or pore complex are preferably variants of SEQ ID NO: 3. The variants in the pore complex may be the same or different. The variants are preferably identical in each pore monomer conjugate in the pore complex of the invention.

### CsgF Peptide

The term “CsgF peptide” preferably defines a CsgF peptide that has been truncated from its C-terminal end (i.e., is an N-terminal fragment). The CsgF peptide may be a fragment of wild-type E. coli CsgF (SEQ ID NO: 5 or SEQ ID NO: 6), or of a wild-type homologue of E. coli CsgF, such as for example, a peptide comprising any one of the amino acid sequences shown in WO 2019/002893 (incorporated by reference herein in its entirety). A CsgF homologue is referred to as a polypeptide that has at least 40%, 50%, 60%, 70%, 80%, 90%, 95% or 99% complete sequence identity to wild-type E. coli CsgF as shown in SEQ ID NO: 6. A CsgF homologue may also referred to as a polypeptide that contains the PFAM domain PF10614, which is characteristic for CsgF-like proteins. A list of presently known CsgF homologues and CsgF architectures can be found at http://pfam.xfam.org//family/PF10614. Mature CsgF (shown in SEQ ID NO:6) can be divided into three main regions: a “CsgF constriction peptide” (FCP), a “neck” region and a “head” region. The “head” region of the CsgF peptide is distinct from a constriction of a pore as described herein. The “head” region of the CsgF peptide may also be referred to as the “C-terminal head domain”. The structure of CsgF is discussed in detail in WO 2019/002893 (incorporated by reference herein in its entirety).

The CsgF peptide used in the pore monomer conjugate of the invention is preferably a truncated CsgF peptide lacking the C-terminal head; lacking the C-terminal head and a part of the neck domain of CsgF (e.g., the truncated CsgF peptide may comprise only a portion of the neck domain of CsgF); or lacking the C-terminal head and neck domains of CsgF. The CsgF peptide may lack part of the CsgF neck domain, e.g., the CsgF peptide may comprise a portion of the neck domain, such as for example, from amino acid residue 36 at the N-terminal end of the neck domain (see SEQ ID:NO:6) (e.g. residues 36-40, 36-41, 36-42, 36-43, 36-45, 36-46 up to residues 36-50 or 36-60 of SEQ ID NO: 6). The CsgF peptide preferably comprises a CsgG-binding region and a region that forms a constriction in the pore. The CsgG-binding region typically comprises residues 1 to 11 and/or 29 to 32 of the CsgF protein (SEQ ID NO: 6 or a homologue from another species) and may include one or more modifications. The region that forms a constriction in the pore typically comprises residues 9 to 28 of the CsgF protein (SEQ ID NO: 6 or a homologue from another species) and may include one or more modifications. Residues 9 to 17 comprise the conserved motif N₉PXFGGXXX₁₇ and form a turn region. Residues 9 to 28 form an alpha-helix. X₁₇ (N17 in SEQ ID NO: 6) forms the apex of the constriction region, corresponding to the narrowest part of the CsgF constriction in the pore. The CsgF constriction region also makes stabilising contacts with the CsgG beta-barrel, primarily at residues 8, 9, 10, 11, 12, 18, 21, 22, 29 and 30 of SEQ ID NO: 6.

The CsgF peptide typically has a length of from 28 to 50 amino acids, such as 29 to 49, 30 to 45 or 32 to 40 amino acids. Preferably the CsgF peptide comprises from 29 to 35 amino acids, or 29 to 45 amino acids. The CsgF peptide comprises all or part of the FCP, which corresponds to residues 1 to 35 of SEQ ID NO: 6. Where the CsgF peptide is shorter that the FCP, the truncation is preferably made at the C-terminal end.

The CsgF peptide may have a length of 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54 or 55 amino acids.

The CsgF peptide may comprise the amino acid sequence of SEQ ID NO: 6 from residue 1 up to any one of residues 25 to 60, such as 27 to 50, for example, 28 to 45 of SEQ ID NO: 6, or the corresponding residues from a homologue of SEQ ID NO: 6, or variant of either thereof. More specifically, the CsgF peptide may comprise residues 1 to 29 of SEQ ID NO: 6, or a homologue or variant thereof.

The CsgF peptide is preferably a truncated CsgF peptide lacking one or more amino acids from CsgF shown in SEQ ID NO: 6. The CsgF peptide is preferably a truncated CsgF peptide lacking a stretch of amino acids starting at any one of positions 15-35 and finishing at position 119 of SEQ ID NO: 6. The CsgF peptide is preferably a truncated CsgF peptide lacking amino acids 15-119, 16-119, 17-119, 18-119, 19-119,20-119, 21-119,22-119, 23-119,24-119, 25-119,26-119, 27-119, 28-119, 29-119, 30-119, 31-119, 32-119, 33-119, 34-119, or 35-119 from SEQ ID NO: 6.

Examples of such CsgF peptides comprises, consist essentially of, or consist of residues 1 to 34 of SEQ ID NO: 6, residues 1 to 30 of SEQ ID NO: 6, residues 1 to 45 of SEQ ID NO: 6, or residues 1 to 35 of SEQ ID NO: 6 and homologues or variants of any thereof.

In the CsgF peptide, one or more residues may be modified. For example, the CsgF peptide may comprise a modification at a position corresponding to one or more of the following positions in SEQ ID NO: 6: G1, T4, F5, R8, N9, N11, F12, N17, A20, N24, A26, Q27 and Q29.

The CsgF peptide may be modified to introduce one or more cysteines, one or more hydrophobic amino acids, one or more charged amino acids, one or more non-native amino acids, one or more polar amino acids, or one or more photoreactive amino acids, for example at a position corresponding to one or more of the following positions in SEQ ID NO: 6: G1, T4, F5, R8, N9, N11, F12, A26 and Q29. Any number and combination of such introductions may be made. The introduction is preferably by substitution or addition.

For example, the CsgF peptide may comprise a modification at a position corresponding to one or more of the following positions in SEQ ID NO: 6: N15, N17, A20, N24 and A28. The CsgF peptide may comprise a modification at a position corresponding to D34 to stabilise the CsgG-CsgF complex. The CsgF peptide may comprise one or more of the substitutions: N15S/A/T/Q/G/L/V/I/F/Y/W/R/K/D/C/E, N17S/A/T/Q/G/L/V/I/F/Y/W/R/K/D/C/E, A20S/T/Q/N/G/L/V/I/F/Y/W/R/K/D/C/E, N24S/T/Q/A/G/L/V/I/F/Y/W/R/K/D/C/E, A28S/T/Q/N/G/L/V/I/F/Y/W/R/K/D/C/E and D34F/Y/W/R/K/N/Q/C/E. The CsgF peptide may, for example, comprise one or more of the following substitutions: G1C, T4C, N17S, and D34Y or D34N.

The CsgF peptide may be produced by cleavage of a longer protein, such as full-length CsgF using an enzyme. Cleavage at a particular site may be directed by modifying the longer protein, such as full-length CsgF, to include an enzyme cleavage site at an appropriate position. Examples of CsgF amino acid sequences that have been modified to include such enzyme cleavage sites are shown in SEQ ID NOs: 56 to 67 of WO 2019/002893 (incorporated by reference herein in its entirety). Following cleavage all or part of the added enzyme cleavage site may be present in the CsgF peptide that associates with CsgG to form a pore. Thus, the CsgF peptide may further comprise all or part of an enzyme cleavage site at its C-terminal end.

Some examples of suitable CsgF peptides are shown in Table 3 of WO 2019/002893 (incorporated by reference herein in its entirety).

The CsgF peptide is preferably a variant of any of the CsgF sequences discussed above, including SEQ ID NO: 6, comprising one or more modifications compared with the comparative sequence. Over the entire length of the amino acid sequence of SEQ ID NO: 6, a variant will preferably be at least 40% homologous to that sequence based on amino acid identity. More preferably, the variant may be at least 45%, at least 50%, at least 55%, at least 60%, at least 65%, at least 70%, at least 75%, at least 80%, at least 85%, at least 90% and more preferably at least 95%, 97% or 99% homologous based on amino acid identity to the amino acid sequence of SEQ ID NO: 6 over the entire sequence. Over the entire length of the amino acid sequence of SEQ ID NO: 6, a variant will preferably be at least 40% identical to that sequence. More preferably, the variant may be at least 45%, at least 50%, at least 55%, at least 60%, at least 65%, at least 70%, at least 75%, at least 80%, at least 85%, at least 90% and more preferably at least 95%, 97% or 99% identical to SEQ ID NO: 6 over the entire sequence. There may be at least 80%, for example at least 85%, 90% or 95%, amino acid identity over a stretch of 100 or more, for example 125, 150, 175 or 200 or more, contiguous amino acids (“hard homology”). These levels of homology/identity equally apply to any of the other CsgF peptides described above.

Any number of the CsgF peptides in the pore or pore complex of the invention, such as 6, 7, 8, 9 or 10, may contain one or more substitutions compared with SEQ ID NO: 6. All six to ten monomers in the pore or pore complex preferably contain one or more substitutions compared with SEQ ID NO: 6. The CsgF peptides in the pore complex may be the same or different. The CsgF peptides are preferably identical in each pore monomer conjugate in the pore complex of the invention.

### Stabilisation and Other Mutations

In the pore complex of the invention, the interaction between the CsgF peptide and the CsgG pore may, for example, be stabilised by hydrophobic interactions and/or electrostatic interactions. These may be interactions between one or more of the following pairs of positions of SEQ ID NO: 6 and SEQ ID NO: 3, respectively: 1 and 153, 4 and 133, 5 and 136, 8 and 187, 8 and 203, 9 and 203, 11 and 142, 11 and 201, 12 and 149, 12 and 203, 26 and 191, and 29 and 144.

The residues in the CsgF peptide and/or the CsgG pore monomer at one or more of the positions listed above may be modified in order to enhance the interaction between CsgG and CsgF in the pore complex. Although the CsgG:CsgF complex is very stable, when CsgF is truncated, the stability of CsgG:CsgF complexes decrease compared to a complex comprising full length CsgF. Therefore, disulfide bonds can be made between CsgG and CsgF to make the complex more stable, for example following introduction of cysteine residues at the positions identified herein. The pore complex can be made in any of the previously mentioned methods and disulfide bond formation can be induced by using oxidising agents (eg: Copper-orthophenanthroline). Other interactions (eg: hydrophobic interactions, charge-charge interactions/electrostatic interactions) can also be used in those positions instead of cysteine interactions.

Unnatural amino acids can also be incorporated in those positions. Covalent bonds may be by via click chemistry. For example, unnatural amino acids with azide or alkyne or with a dibenzocyclooctyne (DBCO) group and/or a bicyclo[6.1.0]nonyne (BCN) group may be introduced at one or more of these positions.

Such stabilising mutations can be combined with any other modifications to CsgG and/or CsgF, for example the modifications disclosed herein.

To facilitate such interactions, one or more non-native or photoreactive amino acids may be included/substituted in the CsgG pore monomer at one or more positions corresponding to one or more of positions 132, 133, 136, 138, 140, 142, 144, 145, 147, 149, 151, 153, 155, 183, 185, 187, 189, 191, 201, 203, 205, 207 and 209 of SEQ ID NO: 3.

To facilitate such interactions, one or more non-native reactive or photoreactive amino acids may be included/substituted at one or more positions corresponding to one or more of positions 1, 4, 5, 8, 9, 11, 12, 26 or 29 of SEQ ID NO: 6.

Preferred exemplary CsgF peptides comprise the following mutations relative to SEQ ID NO: 6: N15X₁/N17X₂/A20X₃/N24X₄/A28X₅/D34X₆, wherein X₁ is N/S/A/T/Q/G/L/V/I/F/Y/W/R/K/D/C/E, X₂ is N/S/A/T/Q/G/L/V/I/F/Y/W/R/K/D/C/E, X₃ is A/S/T/Q/N/G/L/V/I/F/Y/W/R/K/D/C/E, X₄ is N/S/T/Q/A/G/L/V/I/F/Y/W/R/K/D/C/E, Xₛ is A/S/T/Q/N/G/L/V/I/F/Y/W/R/K/D/C/E and X₆ is D/F/Y/W/R/K/N/Q/C/E. The mutations at positions N15, N17, A20, N24 and A28 are constriction mutations and the mutation at position 34 affects the interaction of CsgF with the bottom of the CsgG pore monomer to stabilise the interaction.

### Constructs

The invention also provides a construct comprising two or more covalently attached pore monomer conjugates of the invention. The construct may comprise 2 or more, 3 or more, 4 or more, 5 or more, 6 or more, 7 or more, 8 or more, 9 or more or 10 or more pore monomer conjugates of the invention. The construct may comprise at least 2, at least 3, at least 4, at least 5, at least 6, at least 7, at least 8, at least 9 or at least 10 pore monomer conjugates of the invention. The two or more pore monomer conjugates may be the same or different. The two or more pore monomer conjugates may differ based on one or more of (a) the sequence of the CsgG pore monomer, (b) the sequence of the CsgF peptide, (c) the linker, (d) the attachment position on the CsgG pore monomer, and (e) the attachment position on the CsgF peptide. The pore monomer conjugates may differ based on (a); (b); (c); (d); (e); (a) and (b); (a) and (c); (a) and (d); (a) and (e); (b) and (c); (b) and (d); (b) and (e); (c) and (d); (c) and (e); (d) and (e); (a), (b) and (c); (a), (b) and (d); (a), (b) and (e); (a), (c) and (d); (a), (c) and (e); (a), (d) and (e); (b), (c) and (d); (b), (c) and (e); (b), (d) and (e); (c), (d) and (e); (a), (b), (c) and (d); (a), (b), (c) and (e); (a), (b), (d) and (e); (a), (c), (d) and (e); (b), (c), (d) and (e); and (a), (b), (c), (d) and (e). The two or more pore monomer conjugates are preferably the same (i.e., identical).

The construct preferably comprises two pore monomer conjugates. The two or more pore monomer conjugates may be the same or different. The two or more pore monomer conjugates are preferably the same (i.e., identical).

The pore monomer conjugates may be genetically fused, optionally via a linker, or chemically fused, for instance via a chemical crosslinker. Methods for covalently attaching monomers are disclosed in WO 2017/149316, WO 2017/149317, and WO 2017/149318 (incorporated herein by reference in their entirety).

The linker is preferably an amino acid sequence and/or a chemical crosslinker. Suitable amino acid linkers, such as peptide linkers, are known in the art. The length, flexibility and hydrophilicity of the amino acid or peptide linker are typically designed such that the CsgF peptide forms a constriction in the pore complex of the invention. Preferred flexible peptide linkers are stretches of 2 to 20, such as 4, 6, 8, 10 or 16, serine and/or glycine amino acids. More preferred flexible linkers include (SG)₁, (SG)₂, (SG)₃, (SG)₄, (SG)₅, (SG)₈, (SG)₁₀, (SG)₁₅ or (SG)₂₀ wherein S is serine and G is glycine. Preferred rigid linkers are stretches of 2 to 30, such as 4, 6, 8, 16 or 24, proline amino acids. More preferred rigid linkers include (P)₁₂ wherein P is proline.

Suitable chemical crosslinkers are well-known in the art. Suitable chemical crosslinkers include, but are not limited to, those including the following functional groups: maleimide, active esters, succinimide, azide, alkyne (such as dibenzocyclooctynol (DIBO or DBCO), difluoro cycloalkynes and linear alkynes), phosphine (such as those used in traceless and non-traceless Staudinger ligations), haloacetyl (such as iodoacetamide), phosgene type reagents, sulfonyl chloride reagents, isothiocyanates, acyl halides, hydrazines, disulfides, vinyl sulfones, aziridines and photoreactive reagents (such as aryl azides, diaziridines).

Reactions between amino acids and functional groups may be spontaneous, such as cysteine/maleimide, or may require external reagents, such as Cu(I) for linking azide and linear alkynes.

Linkers can comprise any molecule that stretches across the distance required. Linkers can vary in length from one carbon (phosgene-type linkers) to many Angstroms. Examples of linker molecules, include but are not limited to, are polyethyleneglycols (PEGs), polypeptides, polysaccharides, deoxyribonucleic acid (DNA), peptide nucleic acid (PNA), threose nucleic acid (TNA), glycerol nucleic acid (GNA), saturated and unsaturated hydrocarbons, polyamides. These linkers may be inert or reactive, in particular they may be chemically cleavable at a defined position, or may be themselves modified with a fluorophore or ligand. The linker is preferably resistant to reducing agents, such as dithiothreitol (DTT), following the attachment, such as covalent attachment, of the CsgF peptide to the CsgG pore monomer.

Preferred crosslinkers include 2,5-dioxopyrrolidin-1-yl 3-(pyridin-2-yldisulfanyl)propanoate, 2,5-dioxopyrrolidin-1-yl 4-(pyridin-2-yldisulfanyl)butanoate and 2,5-dioxopyrrolidin-1-yl 8-(pyridin-2-yldisulfanyl)octananoate, di-maleimide PEG 1k, di-maleimide PEG 3.4k, di-maleimide PEG 5k, di-maleimide PEG 10k, bis(maleimido)ethane (BMOE), bis-maleimidohexane (BMH), 1,4-bis-maleimidobutane (BMB), 1,4 bis-maleimidyl-2,3-dihydroxybutane (BMDB), BM[PEO]2 (1,8-bis-maleimidodiethyleneglycol), BM[PEO]3 (1,11-bis-maleimidotriethylene glycol), tris[2-maleimidoethyl]amine (TMEA), DTME dithiobismaleimidoethane, bis-maleimide PEG3, bis-maleimide PEG11, DBCO-maleimide, DBCO-PEG4-maleimide, DBCO-PEG4-NH2, DBCO-PEG4-NHS, DBCO-NHS, DBCO-PEG-DBCO 2.8 kDa, DBCO-PEG-DBCO 4.0 kDa, DBCO-15 atoms-DBCO, DBCO-26 atoms-DBCO, DBCO-35 atoms-DBCO, DBCO-PEG4-S—S-PEG3-biotin, DBCO—S—S-PEG3-biotin, DBCO—S—S—PEG11-biotin, (succinimidyl 3-(2-pyridyldithio)propionate (SPDP) and maleimide-PEG(2 kDa)-maleimide (ALPHA,OMEGA-BIS-MALEIMIDO POLY(ETHYLENE GLYCOL)). The most preferred crosslinker is maleimide-propyl-SRDFWRS-(1,2-diaminoethane)-propyl-maleimide.

The linker is preferably resistant to dithiothreitol (DTT). Suitable linkers include, but are not limited to, iodoacetamide-based and maleimide-based linkers.

The pore monomer conjugates may be connected using two or more linkers each comprising a hybridizable region and a group capable of forming a covalent bond. The hybridizable regions in the linkers hybridize and link the CsgG pore monomer and CsgF peptide. The linked CsgG pore monomer and CsgF peptide are then coupled via the formation of covalent bonds between the groups. Any of the specific linkers disclosed in WO 2010/086602 (incorporated herein by reference in its entirety) may be used in accordance with the invention.

The linkers may be labelled. Suitable labels include, but are not limited to, fluorescent molecules (such as Cy3 or AlexaFluor®555), radioisotopes, e.g. ¹²⁵I, ³⁵S, ³²P, enzymes, antibodies, antigens, polynucleotides and ligands such as biotin. Such labels allow the amount of linker to be quantified. The label could also be a cleavable purification tag, such as biotin, or a specific sequence to show up in an identification method, such as a peptide that is not present in the protein itself, but that is released by trypsin digestion.

A preferred method of connecting the pore monomer conjugates is via cysteine linkage. This can be mediated by a bi-functional chemical crosslinker or by an amino acid linker with a terminal presented cysteine residue.

Another preferred method of attachment via 4-azidophenylalanine or Faz linkage. This can be mediated by a bi-functional chemical linker or by a polypeptide linker with a terminal presented 4-azidophenylalanine or Faz residue. Additional suitable linkers are discussed in more detail below.

### Pore Complexes of the Invention

The term “pore complex”, or “complex pore”, as used interchangeably herein, refer to an oligomeric pore complex comprising at least one pore monomer conjugate of the invention (including, e.g., one or more pore monomer conjugates such as two or more pore monomer conjugates, three or more pore monomer conjugates etc.). The pore complex of the invention has the features of a biological pore, i.e., it has a typical protein structure and defines a channel. When the pore complex is provided in an environment having membrane components, membranes, cells, or an insulating layer, the pore complex will insert in the membrane or the insulating layer and form a “transmembrane pore complex”.

The CsgG part of the pore complex of the invention (i.e., the part formed from the at least one CsgG pore monomer in the at least one conjugate of the invention) preferably has or comprises any of the structures and/or dimensions of the CsgG pores discussed above. The CsgG constriction in the pore complex of the invention preferably has or comprises any of the constriction diameters described above.

The at least one CsgF peptide (in the at least one pore monomer conjugate or construct) preferably forms a constriction in the pore complex. The at least one CsgF peptide is preferably inserted into the lumen of the pore complex. The invention relates to CsgG pores complexed with a CsgF peptide that introduces an additional channel constriction in the pore complex and surprisingly results in an increased current range and increased signal-to-noise ratio (SNR). The additional constriction introduced by complex formation with the CsgF peptides expands the contact surface with passing analytes and can act as a second constriction for analyte detection and characterization. Pores comprising the pore monomer conjugates of the invention can improve the characterisation of analytes, such as polynucleotides, providing a more discriminating direct relationship between the observed current as the polynucleotide moves through the pore. In particular, by having two stacked constrictions spaced at a defined distance, the pore complex may facilitate characterization of polynucleotides that contain at least one homopolymeric stretch, e.g., several consecutive copies of the same nucleotide that otherwise exceed the interaction length of the single CsgG constriction. Additionally, by having two stacked constrictions at a defined distance, small molecule analytes including organic or inorganic drugs and pollutants passing through the pore complex will consecutively pass the two constrictions. The chemical nature of either constriction can be independently modified, each giving unique interaction properties with the analyte, thus providing additional discriminating power during analyte detection.

The CsgF constriction formed in the pore complex preferably has a diameter in the range of from about 5 to about 20 Å, such as from about 7 to about 18 Å, from about 10 Å to about 15 Å or from about 11 to about 12 Å. The additional CsgF peptide constriction may be about 10 nm or less, such as about 5 nm or less, such as about 1, 2, 3, 4, 5, 6, 7, 8, 9 nm from the constriction of the CsgG pore. Distances between the CsgF peptide and CsgG pore monomer are also discussed above with reference to the pore monomer conjugates of the invention.

The pore complex or transmembrane pore complex of the invention includes a pore complex with two constrictions, i.e., two channel constrictions positioned in such a way that one constriction does not interfere in the accuracy of the other constriction. Said pore complexes may include any of the mutations, CsgG pore monomers or CsgF peptides are described in WO 2016/034591, WO 2017/149316, WO 2017/149317, WO 2019/002893, WO 2017/149318, WO 2018/211241, and WO 2019/002893 (herein all incorporated by reference in their entirety). The pore complex or transmembrane pore complex of the invention includes a pore complex with one constriction. For instance, the constriction may be removed from the CsgG pore monomer in the conjugate of the invention such that the pore complex of the invention only contains one constriction provided by the CsgF peptide. The invention provides a pore complex comprising at least one pore monomer conjugate of the invention. The pore complex typically comprises at least 6, 7, 8, 9 or 10 pore monomer conjugates of the invention. The pore complex preferably comprises 8 or 9 pore monomer conjugates of the invention. The pore monomer conjugates are typically the same (i.e., identical).

The pore complex is preferably a homooligomer comprising 6 to 10, such as 6, 7, 8, 9 or 10, pore monomer conjugates of the invention. The pore monomer conjugates are typically identical. The pore complex preferably comprises 8 or 9 identical pore monomer conjugates of the invention. The pore monomer conjugates may be any of those discussed above.

The invention provides a pore complex comprising at least one construct of the invention. The pore complex typically comprises at least 1, 2, 3, 4 or 5 constructs of the invention. The pore complex comprises sufficient CsgG pore monomers to form a pore. For instance, an octameric pore may comprise (a) four constructs each comprising two pore monomer conjugates, (b) two constructs each comprising four pore monomer conjugates, (c) one construct comprising two pore monomer conjugates and six pore monomer conjugates that do not form part of a construct, (d) three constructs comprising two pore monomer conjugates and two pore monomer conjugates that do not form part of a construct, and (e) combinations thereof. Same and additional possibilities are provided for a nonameric pore for instance. Other combinations of constructs and monomers can be envisaged by the skilled person. One or more constructs of the invention may be used to form a pore complex for characterising, such as sequencing, polynucleotides. The pore complex preferably comprises 4 constructs of the invention each of which comprises two pore monomer conjugates. The constructs are typically the same (i.e., identical).

The pore complex is preferably a homooligomer comprising 1-5, such as 1, 2, 3, 4, 5, constructs of the invention. The constructs are typically the same (i.e., identical). The pore complex preferably comprises 4 identical constructs of the invention each of which comprises two pore monomer conjugate. The constructs may be any of those discussed above.

The CsgG pore monomers in the CsgG pore are preferably all approximately the same length or are the same length. The barrels of the CsgG pore monomers of the invention in the pore are preferably approximately the same length or are the same length. Length may be measured in number of amino acids and/or units of length.

The pore complex of the invention may be isolated, substantially isolated, purified or substantially purified. A pore complex of the invention is isolated or purified if it is completely free of any other components, such as lipids or other pores. A pore complex is substantially isolated if it is mixed with carriers or diluents which will not interfere with its intended use. For instance, a pore complex is substantially isolated or substantially purified if it is present in a form that comprises less than 10%, less than 5%, less than 2% or less than 1% of other components, such as block copolymers, lipids, or other pores. Alternatively, a pore complex of the invention may be present in a membrane. Suitable membranes are discussed below.

A pore complex of the invention may be present as an individual or single pore complex. Alternatively, a pore complex of the invention may be present in a homologous or heterologous population of two or more pore complexes or pores. Other formats involving the pore complexes of the invention are discussed in more detail below.

### Multimeric Pore Complexes

The invention also provides a pore multimer comprising two or more pores, wherein at least one of the pores is a pore complex of the invention. The multimer may comprise any number of pores, such as 3, 4, 5, 6, 7 or 8 or more pores. Any number of the pores in the multimer, including all of them, may be a pore complex of the invention.

The pore multimer may be a double pore complex comprising a first pore complex of the invention and a second pore or complex. The second pore or complex is typically derived from CsgG. The second pore complex may be a complex of the invention. Both the first pore complex and the second pore complex are preferably pore complexes of the invention. In the double pore complex, the first pore complex may be attached to the second pore (complex) by hydrophobic interactions and/or by one or more disulfide bonds. One or more, such as 2, 3, 4, 5, 6, 8, 9, for example all, of the monomers in the first pore complex and/or the second pore (complex) may be modified to enhance such interactions. This may be achieved in any suitable way. Particular methods of forming double pores from CsgG-derived pores are described in WO 2019/002893 (incorporated by reference herein in its entirety).

The pore multimer of the invention may be isolated, substantially isolated, purified or substantially purified. Such terms are defined above with reference to the pore complexes of the invention.

### Membrane Embodiments

The invention also provides a pore complex of the invention or a pore multimer of the invention which is comprised in a membrane. The invention also provides a membrane comprising a pore complex of the invention or a pore multimer of the invention. These products are directly applicable for use in molecular sensing, such as analyte characterisation and polynucleotide sequencing. Suitable membranes are discussed in more detail below.

### Method for Making Modified Proteins

Methods for introducing or substituting non-naturally occurring amino acids in CsgG pore monomers and CsgF peptides are also well known in the art and described in WO 2019/002893 (incorporated by reference herein in its entirety). The proteins may be modified to assist their identification or purification, for example by the addition of a streptavidin tag or by the addition of a signal sequence to promote their secretion from a cell where the monomer does not naturally contain such a sequence. The proteins may also be produced using D-amino acids or a mixture of L-amino acids and D-amino acids. This is conventional in the art for producing such proteins or peptides.

The CsgG pore monomer, the CsgF peptide, the pore monomer conjugate, the construct, the pore complex, or the pore multimer (i.e., any protein of the invention) may be chemically modified. The protein can be chemically modified in any way and at any site. The protein may be chemically modified by attachment of a molecule to one or more cysteines (cysteine linkage), attachment of a molecule to one or more lysines, attachment of a molecule to one or more non-natural amino acids, enzyme modification of an epitope or modification of a terminus. Suitable methods for carrying out such modifications are well-known in the art. The protein may be chemically modified by the attachment of any molecule, such as a dye or a fluorophore.

The protein may be chemically modified with a molecular adaptor that facilitates the interaction between a pore comprising the monomer and a target nucleotide or target polynucleotide sequence. Suitable adaptors, including a cyclic molecule, a cyclodextrin, a species that is capable of hybridization, a DNA binder or interchelator, a peptide or peptide analogue, a synthetic polymer, an aromatic planar molecule, a small positively charged molecule or a small molecule capable of hydrogen-bonding, are described in WO 2019/002893 (incorporated by reference herein in its entirety). The molecular adaptor may be attached using any of the methods and linkers discussed above.

The protein may be attached to a polynucleotide binding protein. This forms a modular sequencing system that may be used in the methods of sequencing of the invention. Polynucleotide binding proteins are discussed below. The protein can be covalently attached to the monomer using any method known in the art. The monomer and protein may be chemically fused or genetically fused. Genetic fusion of a monomer to a polynucleotide binding protein is discussed in WO 2010/004265 (incorporated herein by reference in its entirety). The polynucleotide binding protein may be attached via cysteine linkage using any method described above.

The polynucleotide binding protein may be attached directly to the protein via one or more linkers. The molecule may be attached to the CsgG pore monomer using the hybridization linkers described in as WO 2010/086602 (incorporated herein by reference in its entirety). Alternatively, peptide linkers may be used. Suitable peptide linkers are discussed above.

Any of the proteins may be modified to assist their identification or purification, for example by the addition of histidine residues (a his tag), aspartic acid residues (an asp tag), a streptavidin tag, a flag tag, a SUMO tag, a GST tag or a MBP tag, or by the addition of a signal sequence to promote their secretion from a cell where the polypeptide does not naturally contain such a sequence. An alternative to introducing a genetic tag is to chemically react a tag onto a native or engineered position on the protein. An example of this would be to react a gel-shift reagent to a cysteine engineered on the outside of the protein. This has been demonstrated as a method for separating hemolysin hetero-oligomers (Chem Biol. 1997 July; 4(7):497-505).

Any of the proteins may be labelled with a revealing label. The revealing label may be any suitable label which allows the protein to be detected. Suitable labels include, but are not limited to, fluorescent molecules, radioisotopes, e.g., 125I, 35S, enzymes, antibodies, antigens, polynucleotides, and ligands such as biotin.

The protein may also contain other non-specific modifications as long as they do not interfere with the function of the protein. A number of non-specific side chain modifications are known in the art and may be made to the side chains of the protein(s). Such modifications include, for example, reductive alkylation of amino acids by reaction with an aldehyde followed by reduction with NaBH4, amidation with methylacetimidate or acylation with acetic anhydride.

Any of the proteins can be produced using standard methods known in the art. Polynucleotide sequences encoding a protein may be derived and replicated using standard methods in the art. Polynucleotide sequences encoding a protein may be expressed in a bacterial host cell using standard techniques in the art. The protein may be produced in a cell by in situ expression of the polypeptide from a recombinant expression vector. The expression vector optionally carries an inducible promoter to control the expression of the polypeptide. These methods are described in Sambrook, J. and Russell, D. (2001). Molecular Cloning: A Laboratory Manual, 3rd Edition. Cold Spring Harbor Laboratory Press, Cold Spring Harbor, NY.

Proteins may be produced in large scale following purification by any protein liquid chromatography system from protein producing organisms or after recombinant expression. Typical protein liquid chromatography systems include FPLC, AKTA systems, the Bio-Cad system, the Bio-Rad BioLogic system, and the Gilson HPLC system.

### Method for Producing Pore Monomer Conjugates

The invention provides methods for producing a pore monomer conjugate of the invention. The method comprises attaching, preferably covalently attaching, the CsgF peptide to the the CsgG pore monomer using a sulfonyl fluoride-containing linker, a sulfonyl triazole-containing linker, a fluorosulfate-containing linker, or fluoroacetamide-containing linker. The method may involve using any of the linkers described above. The linker may attach or covalently attach the CsgF peptide to the CsgG pore monomer at any of the positions discussed above with reference to the pore monomer conjugates of the invention.

Alternatively, the method comprises attaching, preferably covalently attaching, the CsgF peptide to a residue in the CsgG pore monomer corresponding to any one of positions 143, 146, 190, 192, 193, 194, 195, 196, 197, 198, 199 or 200 in SEQ ID NO: 3. The method may involve using any of the reactive groups and/or linkers described above.

The methods typically comprise contacting the CsgF peptide and the CsgG pore monomer with the linker. The components may be contacted with the linker in any order, such as CsgF peptide first and then the CsgG pore monomer, the CsgG pore monomer first and then the CsgF peptide or both components at the same time. The linker is preferably attached to the CsgF peptide or the CsgG pore monomer first and then attached to the other component of the conjugate. The method preferably comprises attaching or covalently attaching the linker to the CsgF peptide and then contacting the linker and CsgF peptide with the CsgG pore monomer under conditions which attaching or covalently attach the CsgF peptide to the CsgG pore monomer by the linker. Such conditions are well known to a person skilled in the art and are discussed in the Examples. The method is typically carried out in vitro as defined below.

Any of the embodiments discussed above with reference to the pore monomer conjugates of the invention equally applies to these methods.

### Method of Producing Pores

The invention also provides methods for producing a pore complex of the invention or a pore multimer of the invention.

The method may involve expressing the pore complex in a host cell. In particular, the method may comprise expressing at least one pore monomer conjugate of the invention or a construct of the invention and sufficient pore monomers or constructs to form the pore complex or the pore multimer in a host cell and allowing the pore complex or pore multimer to form in the host cell. The sufficient pore monomers or constructs are preferably sufficient pore monomer conjugates of the invention or sufficient constructs of the invention. The numbers of CsgG pore monomers, pore monomer conjugates or constructs needed to form the pore complexes of the invention or pore multimers of the invention are discussed above. Suitable host cells and expression systems are known in the art and are discussed in the Examples.

The method may involve forming the pore complex in a non-cellular or in vitro context. In particular, the method may comprise contacting at least one pore monomer conjugate of the invention or a construct of the invention with sufficient pore monomers or constructs in vitro and allowing the formation of the pore complex or pore multimer. The pore monomer conjugate or the construct may be produced separately by in vitro translation and transcription (IVTT) and then incubated with the sufficient pore monomers or constructs. The sufficient pore monomers or constructs are preferably sufficient pore monomer conjugates of the invention or sufficient constructs of the invention. The numbers of CsgG pore monomers, pore monomer conjugates or constructs needed to form the pore complexes of the invention or pore multimers of the invention are discussed above. The method may be conducted in an “in vitro system”, which refers to a system comprising at least the necessary components and environment to execute said method, and makes use of biological molecules, organisms, a cell (or part of a cell) outside of their normal naturally occurring environment, permitting a more detailed, more convenient, or more efficient analysis than can be done with whole organisms. An in vitro system may also comprise a suitable buffer composition provided in a test tube, wherein said protein components to form the complex have been added. A person skilled in the art is aware of the options to provide said system.

Some or all of the components of the pore complex or pore multimer may be tagged to facilitate purification. Purification can also be performed when the components are untagged. Methods known in the art (e.g., ion exchange, gel filtration, hydrophobic interaction column chromatography etc.) can be used alone or in different combinations to purify the components of the pore.

The pore complex or pore multimer can be made prior to insertion into a membrane or after insertion of the components into a membrane.

Methods for making the pores and complexes of the invention and ways of tagging them are disclosed in WO 2016/034591, WO 2017/149316, WO 2017/149317 and, WO 2017/149318, WO 2018/211241, and WO 2019/002893 (all incorporated by reference herein in their entirety).

### Methods of Characterising an Analyte

The invention provides a method of determining the presence, absence or one or more characteristics of a target analyte. The method involves contacting the target analyte with a pore complex of the invention or pore multimer of the invention such that the target analyte moves with respect to, such as into or through, the pore complex or pore multimer and taking one or more measurements as the analyte moves with respect to the pore complex or pore multimer and thereby determining the presence, absence or one or more characteristics of the analyte. The target analyte may also be called the template analyte or the analyte of interest.

The pore complex of the invention or the pore multimer of the invention may be any of those discussed above.

The method is for determining the presence, absence or one or more characteristics of a target analyte. The method may be for determining the presence, absence or one or more characteristics of at least one analyte. The method may concern determining the presence, absence or one or more characteristics of two or more analytes. The method may comprise determining the presence, absence or one or more characteristics of any number of analytes, such as 2, 5, 10, 15, 20, 30, 40, 50, 100 or more analytes. Any number of characteristics of the one or more analytes may be determined, such as 1, 2, 3, 4, 5, 10 or more characteristics.

The binding of a molecule in the channel of the pore complex or pore multimer, or in the vicinity of either opening of the channel will have an effect on the open-channel ion flow through the pore complex or pore multimer, which is the essence of “molecular sensing”. In a similar manner to the nucleic acid sequencing application, variation in the open-channel ion flow can be measured using suitable measurement techniques by the change in electrical current (for example, WO 2000/28312 and D. Stoddart et al., Proc. Natl. Acad. Sci., 2010, 106, 7702-7 or WO 2009/077734; all incorporated herein by reference in their entirety). The degree of reduction in ion flow, as measured by the reduction in electrical current, is related to the size of the obstruction within, or in the vicinity of, the pore. Binding of a molecule of interest, also referred to as an “analyte”, in or near the pore therefore provides a detectable and measurable event, thereby forming the basis of a “biological sensor”. Suitable molecules for nanopore sensing include nucleic acids; proteins; peptides; polysaccharides and small molecules (refers here to a low molecular weight (e.g., <900 Da or <500 Da) organic or inorganic compound) such as pharmaceuticals, toxins, cytokines, and pollutants. Detecting the presence of biological molecules finds application in personalised drug development, medicine, diagnostics, life science research, environmental monitoring and in the security and/or the defence industry.

The pore complex or pore multimer may serve as a molecular or biological sensor. The analyte molecule that is to be detected may bind to either face of the channel, or within the lumen of the channel itself. The position of binding may be determined by the size of the molecule to be sensed.

The target analyte is preferably a metal ion, an inorganic salt, a polymer, an amino acid, a peptide, a polypeptide, a protein, a nucleotide, an oligonucleotide, a polynucleotide, a monosaccharide, an oligosaccharide, a polysaccharide, a dye, a bleach, a pharmaceutical, a diagnostic agent, a recreational drug, an explosive, a toxic compound, or an environmental pollutant. The analyte may comprise two or more different molecules, such as a peptide and a polypeptide. The method may concern determining the presence, absence or one or more characteristics of two or more analytes of the same type, such as two or more proteins, two or more nucleotides or two or more pharmaceuticals. Alternatively, the method may concern determining the presence, absence or one or more characteristics of two or more analytes of different types, such as one or more proteins, one or more nucleotides and one or more pharmaceuticals.

The target analyte can be secreted from cells. Alternatively, the target analyte can be an analyte that is present inside cells such that the analyte must be extracted from the cells before the method can be carried out.

The pore complex or pore multimer may be modified via recombinant or chemical methods to increase the strength of binding, the position of binding, or the specificity of binding of the molecule to be sensed. Typical modifications include addition of a specific binding moiety complimentary to the structure of the molecule to be sensed. Where the analyte molecule comprises a nucleic acid, this binding moiety may comprise a cyclodextrin or an oligonucleotide; for small molecules this may be a known complimentary binding region, for example the antigen binding portion of an antibody or of a non-antibody molecule, including a single chain variable fragment (scFv) region or an antigen recognition domain from a T-cell receptor (TCR); or for proteins, it may be a known ligand of the target protein. In this way the pore complex or pore multimer may be rendered capable of acting as a molecular sensor for detecting presence in a sample of suitable antigens (including epitopes) that may include cell surface antigens, including receptors, markers of solid tumours or haematologic cancer cells (e.g. lymphoma or leukaemia), viral antigens, bacterial antigens, protozoal antigens, allergens, allergy related molecules, albumin (e.g. human, rodent, or bovine), fluorescent molecules (including fluorescein), blood group antigens, small molecules, drugs, enzymes, catalytic sites of enzymes or enzyme substrates, and transition state analogues of enzyme substrates. As described above, modifications may be achieved using known genetic engineering and recombinant DNA techniques. The positioning of any adaptation would be dependent on the nature of the molecule to be sensed, for example, the size, three-dimensional structure, and its biochemical nature. The choice of adapted structure may make use of computational structural design. Determination and optimization of protein-protein interactions or protein-small molecule interactions can be investigated using technologies such as a BIAcore® which detects molecular interactions using surface plasmon resonance (BIAcore, Inc., Piscataway, NJ; see also www.biacore.com).

The analyte is preferably an amino acid, a peptide, a polypeptides, or protein. The amino acid, peptide, polypeptide, or protein can be naturally occurring or non-naturally occurring. The polypeptide or protein can include within them synthetic or modified amino acids. Several different types of modification to amino acids are known in the art. Suitable amino acids and modifications thereof are above. It is to be understood that the target analyte can be modified by any method available in the art.

The analyte is preferably a polynucleotide, such as a nucleic acid, which is defined as a macromolecule comprising two or more nucleotides. Nucleic acids are particularly suitable for nanopore sequencing. The naturally occurring nucleic acid bases in DNA and RNA may be distinguished by their physical size. As a nucleic acid molecule, or individual base, passes through the channel of a nanopore, the size differential between the bases causes a directly correlated reduction in the ion flow through the channel. The variation in ion flow may be recorded. Suitable electrical measurement techniques for recording ion flow variations are discussed above. Through suitable calibration, the characteristic reduction in ion flow can be used to identify the particular nucleotide and associated base traversing the channel in real-time. In typical nanopore nucleic acid sequencing, the open-channel ion flow is reduced as the individual nucleotides of the nucleic sequence of interest sequentially pass through the channel of the nanopore due to the partial blockage of the channel by the nucleotide. It is this reduction in ion flow that is measured using the suitable recording techniques described above. The reduction in ion flow may be calibrated to the reduction in measured ion flow for known nucleotides through the channel resulting in a means for determining which nucleotide is passing through the channel, and therefore, when done sequentially, a way of determining the nucleotide sequence of the nucleic acid passing through the nanopore. For the accurate determination of individual nucleotides, it has typically required for the reduction in ion flow through the channel to be directly correlated to the size of the individual nucleotide passing through the constriction. It will be appreciated that sequencing may be performed upon an intact nucleic acid polymer that is ‘threaded’ through the pore via the action of an associated polymerase, for example. Alternatively, sequences may be determined by passage of nucleotide triphosphate bases that have been sequentially removed from a target nucleic acid in proximity to the pore (see for example WO 2014/187924 incorporated herein by reference in its entirety).

The polynucleotide or nucleic acid may comprise any combination of any nucleotides. The nucleotides can be naturally occurring or artificial. One or more nucleotides in the polynucleotide can be oxidized or methylated. One or more nucleotides in the polynucleotide may be damaged. For instance, the polynucleotide may comprise a pyrimidine dimer. Such dimers are typically associated with damage by ultraviolet light and are the primary cause of skin melanomas. One or more nucleotides in the polynucleotide may be modified, for instance with a label or a tag, for which suitable examples are known by a skilled person. The polynucleotide may comprise one or more spacers. A nucleotide typically contains a nucleobase, a sugar and at least one phosphate group. The nucleobase and sugar form a nucleoside. The nucleobase is typically heterocyclic. Nucleobases include, but are not limited to, purines and pyrimidines and more specifically adenine (A), guanine (G), thymine (T), uracil (U) and cytosine (C). The sugar is typically a pentose sugar. Nucleotide sugars include, but are not limited to, ribose and deoxyribose. The sugar is preferably a deoxyribose. The polynucleotide preferably comprises the following nucleosides: deoxyadenosine (dA), deoxyuridine (dU) and/or thymidine (dT), deoxyguanosine (dG) and deoxycytidine (dC). The nucleotide is typically a ribonucleotide or deoxyribonucleotide. The nucleotide typically contains a monophosphate, diphosphate, or triphosphate. The nucleotide may comprise more than three phosphates, such as 4 or 5 phosphates. Phosphates may be attached on the 5′ or 3′ side of a nucleotide. The nucleotides in the polynucleotide may be attached to each other in any manner. The nucleotides are typically attached by their sugar and phosphate groups as in nucleic acids. The nucleotides may be connected via their nucleobases as in pyrimidine dimers. The polynucleotide may be single stranded or double stranded. At least a portion of the polynucleotide is preferably double stranded. The polynucleotide is most preferably ribonucleic nucleic acid (RNA) or deoxyribonucleic acid (DNA). In particular, said method using a polynucleotide as an analyte alternatively comprises determining one or more characteristics selected from (i) the length of the polynucleotide, (ii) the identity of the polynucleotide, (iii) the sequence of the polynucleotide, (iv) the secondary structure of the polynucleotide and (v) whether or not the polynucleotide is modified.

The polynucleotide can be any length (i). For example, the polynucleotide can be at least 10, at least 50, at least 100, at least 150, at least 200, at least 250, at least 300, at least 400 or at least 500 nucleotides or nucleotide pairs in length. The polynucleotide can be 1000 or more nucleotides or nucleotide pairs, 5000 or more nucleotides or nucleotide pairs in length or 100000 or more nucleotides or nucleotide pairs in length. Any number of polynucleotides can be investigated. For instance, the method may concern characterising 2, 3, 4, 5, 6, 7, 8, 9, 10, 20, 30, 50, 100 or more polynucleotides. If two or more polynucleotides are characterised, they may be different polynucleotides or two instances of the same polynucleotide. The polynucleotide can be naturally occurring or artificial. For instance, the method may be used to verify the sequence of a manufactured oligonucleotide. The method is typically carried out in vitro.

Nucleotides can have any identity (ii), and include, but are not limited to, adenosine monophosphate (AMP), guanosine monophosphate (GMP), thymidine monophosphate (TMP), uridine monophosphate (UMP), 5-methylcytidine monophosphate, 5-hydroxymethylcytidine monophosphate, cytidine monophosphate (CMP), cyclic adenosine monophosphate (cAMP), cyclic guanosine monophosphate (cGMP), deoxyadenosine monophosphate (dAMP), deoxyguanosine monophosphate (dGMP), deoxythymidine monophosphate (dTMP), deoxyuridine monophosphate (dUMP), deoxycytidine monophosphate (dCMP) and deoxymethylcytidine monophosphate. The nucleotides are preferably selected from AMP, TMP, GMP, CMP, UMP, dAMP, dTMP, dGMP, dCMP and dUMP. A nucleotide may be abasic (i.e., lack a nucleobase). A nucleotide may also lack a nucleobase and a sugar (i.e., is a C3 spacer). The sequence of the nucleotides (iii) is determined by the consecutive identity of following nucleotides attached to each other throughout the polynucleotide strain, in the 5′ to 3′ direction of the strand.

The pore complexes and pore multimers of the invention are particularly useful in analysing homopolymers. For example, they may be used to determine the sequence of a polynucleotide comprising two or more, such as at least 3, 4, 5, 6, 7, 8, 9 or 10, consecutive nucleotides that are identical. For example, they may be used to sequence a polynucleotide comprising a polyA, polyT, polyG and/or polyC region.

The CsgG pore constriction is made of the residues at the 51, 55 and 56 positions of SEQ ID NO: 3. The constriction of CsgG and its constriction mutants are generally sharp. When DNA is passing through the constriction, interactions of approximately 5 bases of DNA with the constriction of the pore at any given time dominate the current signal. Although these sharper constrictions are very good in reading mixed sequence regions of DNA (when A, T, G and C are mixed), the signal becomes flat and lack information when there is a homopolymeric region within the DNA (eg: polyT, polyG, polyA, polyC). Because 5 bases dominate the signal of the CsgG and its constriction mutants, it's difficult to discriminate photopolymers longer than 5 without using additional dwell time information. However, if DNA is passing through a second constriction formed by the CsgF peptide, more DNA bases will interact with the combined constrictions, increasing the length of the homopolymers that can be discriminated.

The movement of the polynucleotide with respect to the pore, such as through the pore, is preferably controlled using a polynucleotide binding protein. Suitable proteins are discussed in more detail below. The invention provides a method for determining the presence, absence or one or more characteristics of a target polynucleotide, comprising the steps of:
(i) contacting the target polynucleotide with a pore complex of the invention or a pore multimer of the invention and a polynucleotide binding protein, such that the polynucleotide binding protein controls the movement of the target analyte moves with respect to, such as through, the pore complex or the pore multimer; and
(ii) taking one or more measurements as the polynucleotide moves with respect to, such as through, the pore complex or the pore multimer and thereby determining the presence, absence or one or more characteristics of the polynucleotide.

In any of the methods, the one or more characteristics of the target analyte are preferably measured by electrical measurement and/or optical measurement. The electrical measurement is a current measurement, an impedance measurement, a tunnelling measurement, or a field effect transistor (FET) measurement. The method preferably comprises measuring the current flowing through the pore complex or the pore multimer as the analyte moves with respect to, such as through, the pore.

General conditions for conducting the methods of the invention are discussed in more detail below with reference to the kits and systems of the invention.

### Polynucleotides of the Invention

The invention also provides a polynucleotide which encodes a pore monomer conjugate of the invention or a construct of the invention. The polynucleotide may be any of those discussed above. The invention also provides an expression vector comprising a polynucleotide of the invention. The invention also provides a host cell comprising a polynucleotide of the invention or a host cell of the invention. Suitable vectors and host cells are known in the art.

### Kits

The invention also provides kits for characterising a target analyte. In one embodiment, the kit comprises (a) a pore complex of the invention or a pore multimer of the invention and (b) the components of a membrane. Suitable membranes and components are discussed below.

In another embodiment, the kit comprises (a) a pore complex of the invention or a pore multimer of the invention and (b) a polynucleotide binding protein. The kit preferably further comprises the components of a membrane. The kit may comprise components of any type of membranes, such as an amphiphilic layer or a triblock copolymer membrane. Preferred polynucleotide binding proteins are polymerases, exonucleases, helicases, and topoisomerases, such as gyrases. Suitable enzymes include, but are not limited to, exonuclease I from E. coli, exonuclease III enzyme from E. coli, RecJ from T. thermophilus and bacteriophage lambda exonuclease, TatD exonuclease and variants thereof. Three subunits comprising the RecJ sequence from T. thermophilus or a variant thereof interact to form a trimer exonuclease. The polymerase may be PyroPhage® 3173 DNA Polymerase (which is commercially available from Lucigen® Corporation), SD Polymerase (commercially available from Bioron®) or variants thereof. The enzyme may be Phi29 DNA polymerase or a variant thereof. The topoisomerase is preferably a member of any of the Moiety Classification (EC) groups 5.99.1.2 and 5.99.1.3.

The enzyme is most preferably derived from a helicase, such as Hel308 Mbu, Hel308 Csy, Hel308 Tga, Hel308 Mhu, TraI Eco, XPD Mbu or a variant thereof. Any helicase may be used in the invention. The helicase may be or be derived from a Hel308 helicase, a RecD helicase, such as TraI helicase or a TrwC helicase, a XPD helicase or a Dda helicase. The helicase may be any of the helicases, modified helicases or helicase constructs disclosed in WO 2013/057495; WO 2013/098562; WO 2013098561; WO 2014/013260; WO 2014/013259; WO 2014/013262 and WO 2015/055981. All of these are incorporated by reference in their entirety.

The kit may further comprise one or more anchors, such as cholesterol, for coupling the target analyte to the membrane. The kit may further comprise one or more polynucleotide adaptors that can be attached to a target polynucleotide to facilitate characterisation of the polynucleotide. The anchor, such as cholesterol, is preferably attached to the polynucleotide adaptor.

The kit may additionally comprise one or more other reagents or instruments which enable any of the embodiments mentioned above to be carried out. Such reagents or instruments include one or more of the following: suitable buffer(s) (aqueous solutions), means to obtain a sample from a subject (such as a vessel or an instrument comprising a needle), means to amplify and/or express polynucleotides or voltage or patch clamp apparatus. Reagents may be present in the kit in a dry state such that a fluid sample resuspends the reagents. The kit may also, optionally, comprise instructions to enable the kit to be used in the method of the invention or details regarding for which organism the method may be used. Finally, the kit may also comprise additional components useful in analyte characterization.

### Apparatus

The invention also provides an apparatus for characterising target analytes in a sample, comprising (a) a plurality of pore complexes of the invention or a plurality of pore multimers of the invention and (b) a plurality of polynucleotide binding proteins. The plurality of pore complexes or plurality of pore multimers may be any of those discussed above.

The invention also provides an apparatus comprising a pore complex of the invention or a pore multimer of the invention inserted into an in vitro membrane.

The invention also provides an apparatus produced by a method comprising: (i) obtaining a pore complex of the invention or a pore multimer of the invention and (ii) contacting the pore complex or pore multimer with an in vitro membrane such that the pore complex or pore multimer is inserted in the in vitro membrane.

Any of the specific embodiments discussed above are equally applicable to the apparatuses of the invention.

### Arrays

The invention also provides an array comprising a plurality of membranes of the invention. Any of the embodiments discussed above with respect to the membranes of the invention equally apply the array of the invention. The array may be set up to perform any of the methods described below.

In a preferred embodiment, each membrane in the array comprises one pore complex or pore multimer. Due to the manner in which the array is formed, for example, the array may comprise one or more membranes that do not comprise a pore complex or pore multimer, and/or one or more membranes that comprise two or more pores complexes or multimers. The array may comprise from about 2 to about 1000, such as from about 10 to about 800, from about 20 to about 600 or from about 30 to about 500 membranes.

### System

The invention provides a system comprising (a) a membrane of the invention or an array of the invention, (b) means for applying a potential across the membrane(s) and (c) means for detecting electrical or optical signals across the membrane(s).

The pores and membranes may be any as described above and below.

In one embodiment, the system further comprises a first chamber and a second chamber, wherein the first and second chambers are separated by the membrane(s). When used to characterise a target analyte, the system may further comprise a target analyte, wherein the target analyte is transiently located within the continuous channel and wherein one end of the target analyte is located in the first chamber and one end of the target analyte is located in the second chamber. The target analyte is preferably a target polypeptide or a target polynucleotide.

In one embodiment, the system further comprises an electrically conductive solution in contact with the pore(s), electrodes providing a voltage potential across the membrane(s), and a measurement system for measuring the current through the pore(s). The voltage applied across the membranes and pore is preferably from +5 V to −5 V, such as −600 mV to +600 mV or −400 mV to +400 mV. The voltage used is preferably in the range 100 mV to 240 mV and more preferably in the range of 120 mV to 220 mV. It is possible to increase discrimination between different amino acids or nucleotides by a pore by using an increased applied potential. Any suitable electrically conductive solution may be used. For example, the solution may comprise charge carriers, such as metal salts, for example alkali metal salt, halide salts, for example chloride salts, such as alkali metal chloride salt. Charge carriers may include ionic liquids or organic salts, for example tetramethyl ammonium chloride, trimethylphenyl ammonium chloride, phenyltrimethyl ammonium chloride, or 1-ethyl-3-methyl imidazolium chloride. In an exemplary system, salt is present in the aqueous solution in the chamber. Potassium chloride (KCl), sodium chloride (NaCl), caesium chloride (CsCl) or a mixture of potassium ferrocyanide and potassium ferricyanide is typically used. KCl, NaCl and a mixture of potassium ferrocyanide and potassium ferricyanide are preferred. The charge carriers may be asymmetric across the membrane. For instance, the type and/or concentration of the charge carriers may be different on each side of the membrane, e.g., in each chamber.

The salt concentration may be at saturation. The salt concentration may be 3 M or lower and is typically from 0.1 to 2.5 M, from 0.3 to 1.9 M, from 0.5 to 1.8 M, from 0.7 to 1.7 M, from 0.9 to 1.6 M or from 1 M to 1.4 M. The salt concentration is preferably from 150 mM to 1 M. The method is preferably carried out using a salt concentration of at least 0.3 M, such as at least 0.4 M, at least 0.5 M, at least 0.6 M, at least 0.8 M, at least 1.0 M, at least 1.5 M, at least 2.0 M, at least 2.5 M or at least 3.0 M. High salt concentrations provide a high signal to noise ratio and allow for currents indicative of the presence of an amino acid or nucleotide to be identified against the background of normal current fluctuations.

A buffer may be present in the electrically conductive solution. Typically, the buffer is phosphate buffer. Other suitable buffers are HEPES and Tris-HCl buffer. The pH of the electrically conductive solution may be from 4.0 to 12.0, from 4.5 to 10.0, from 5.0 to 9.0, from 5.5 to 8.8, from 6.0 to 8.7 or from 7.0 to 8.8 or 7.5 to 8.5. The pH used is preferably about 7.5.

The system may be comprised in an apparatus. The apparatus may be any conventional apparatus for analyte analysis, such as an array or a chip. The apparatus is preferably set up to carry out the disclosed method. For example, the apparatus may comprise a chamber comprising an aqueous solution and a barrier that separates the chamber into two sections. The barrier typically has an aperture in which the membrane(s) containing the pore(s) are formed. Alternatively, the barrier forms the membrane in which the pore is present.

The apparatus may also comprise an electrical circuit capable of applying a potential and measuring an electrical signal across the membrane and pore.

The apparatus may be any of those described in WO 2008/102120, WO 2009/077734, WO 2010/122293, WO 2011/067559, or WO 00/28312 (all incorporated herein by reference in their entirety).

### Membrane

Any suitable membrane may be used in the system. The membrane is preferably an amphiphilic layer. An amphiphilic layer is a layer formed from amphiphilic molecules, such as phospholipids, which have both hydrophilic and lipophilic properties. The amphiphilic molecules may be synthetic or naturally occurring. Non-naturally occurring amphiphiles and amphiphiles which form a monolayer are known in the art and include, for example, block copolymers (Gonzalez-Perez et al., Langmuir, 2009, 25, 10447-10450). Block copolymers are polymeric materials in which two or more monomer sub-units that are polymerized together to create a single polymer chain. Block copolymers typically have properties that are contributed by each monomer sub-unit. However, a block copolymer may have unique properties that polymers formed from the individual sub-units do not possess. Block copolymers can be engineered such that one of the monomer sub-units is hydrophobic (i.e., lipophilic), whilst the other sub-unit(s) are hydrophilic whilst in aqueous media. In this case, the block copolymer may possess amphiphilic properties and may form a structure that mimics a biological membrane. The block copolymer may be a diblock (consisting of two monomer sub-units) but may also be constructed from more than two monomer sub-units to form more complex arrangements that behave as amphipiles. The copolymer may be a triblock, tetrablock or pentablock copolymer. The membrane is preferably a triblock copolymer membrane.

The membrane is most preferably one of the membranes disclosed in International Application No. WO 2014/064443 or WO 2014/064444.

The amphiphilic molecules may be chemically modified or functionalised to facilitate coupling of the polynucleotide. The amphiphilic layer may be a monolayer or a bilayer. The amphiphilic layer is typically planar. The amphiphilic layer may be curved. The amphiphilic layer may be supported.

Amphiphilic membranes are typically naturally mobile, essentially acting as two-dimensional fluids with lipid diffusion rates of approximately 10⁻⁸ cm s⁻¹. This means that the pore and coupled polynucleotide can typically move within an amphiphilic membrane.

The membrane may be a lipid bilayer. Lipid bilayers are models of cell membranes and serve as excellent platforms for a range of experimental studies. For example, lipid bilayers can be used for in vitro investigation of membrane proteins by single-channel recording. Alternatively, lipid bilayers can be used as biosensors to detect the presence of a range of substances. The lipid bilayer may be any lipid bilayer. Suitable lipid bilayers include, but are not limited to, a planar lipid bilayer, a supported bilayer, or a liposome. The lipid bilayer is preferably a planar lipid bilayer. Suitable lipid bilayers are disclosed in WO 2008/102121, WO 2009/077734, and WO 2006/100484 (all incorporated herein by reference in their entirety).

The membrane preferably comprises a solid-state layer. Solid state layers can be formed from both organic and inorganic materials including, but not limited to, microelectronic materials, insulating materials such as Si₃N₄, Al₂O₃, and SiO, organic and inorganic polymers such as polyamide, plastics such as Teflon® or elastomers such as two-component addition-cure silicone rubber, and glasses. The solid-state layer may be formed from graphene. Suitable graphene layers are disclosed in WO 2009/035647 (incorporated herein by reference in its entirety). If the membrane comprises a solid-state layer, the pore is typically present in an amphiphilic membrane or layer contained within the solid-state layer, for instance within a hole, well, gap, channel, trench or slit within the solid-state layer. The skilled person can prepare suitable solid state/amphiphilic hybrid systems. Suitable systems are disclosed in WO 2009/020682 and WO 2012/005857 (both incorporated herein by reference in their entirety). Any of the amphiphilic membranes or layers discussed above may be used.

The method is typically carried out using (i) an artificial amphiphilic layer comprising a pore, (ii) an isolated, naturally occurring lipid bilayer comprising a pore, or (iii) a cell having a pore inserted therein. The method is typically carried out using an artificial amphiphilic layer, such as an artificial triblock copolymer layer. The layer may comprise other transmembrane and/or intramembrane proteins as well as other molecules in addition to the pore. Suitable apparatus and conditions are discussed below. The method of the invention is typically carried out in vitro.

SEQUENCE LISTING

SEQ ID NO: 1 (>P0AEA2; coding sequence for WT CsgG from E. coli K12)
ATGCAGCGCTTATTTCTTTTGGTTGCCGTCATGTTACTGAGCGGATGCTTAACCGCCC
CGCCTAAAGAAGCCGCCAGACCGACATTAATGCCTCGTGCTCAGAGCTACAAAGAT
TTGACCCATCTGCCAGCGCCGACGGGTAAAATCTTTGTTTCGGTATACAACATTCAG
GACGAAACCGGGCAATTTAAACCCTACCCGGCAAGTAACTTCTCCACTGCTGTTCCG
CAAAGCGCCACGGCAATGCTGGTCACGGCACTGAAAGATTCTCGCTGGTTTATACCG
CTGGAGCGCCAGGGCTTACAAAACCTGCTTAACGAGCGCAAGATTATTCGTGCGGC
ACAAGAAAACGGCACGGTTGCCATTAATAACCGAATCCCGCTGCAATCTTTAACGG
CGGCAAATATCATGGTTGAAGGTTCGATTATCGGTTATGAAAGCAACGTCAAATCTG
GCGGGGTTGGGGCAAGATATTTTGGCATCGGTGCCGACACGCAATACCAGCTCGAT
CAGATTGCCGTGAACCTGCGCGTCGTCAATGTGAGTACCGGCGAGATCCTTTCTTCG
GTGAACACCAGTAAGACGATACTTTCCTATGAAGTTCAGGCCGGGGTTTTCCGCTTT
ATTGACTACCAGCGCTTGCTTGAAGGGGAAGTGGGTTACACCTCGAACGAACCTGTT
ATGCTGTGCCTGATGTCGGCTATCGAAACAGGGGTCATTTTCCTGATTAATGATGGT
ATCGACCGTGGTCTGTGGGATTTGCAAAATAAAGCAGAACGGCAGAATGACATTCT
GGTGAAATACCGCCATATGTCGGTTCCACCGGAATCCTGA
SEQ ID NO: 2 (>P0AEA2 (1:277); WT Pro-CsgG from E. coli K12)
MQRLFLLVAVMLLSGCLTAPPKEAARPTLMPRAQSYKDLTHLPAPTGKIFVSVYNIQDE
TGQFKPYPASNFSTAVPQSATAMLVTALKDSRWFIPLERQGLQNLLNERKIIRAAQENGT
VAINNRIPLQSLTAANIMVEGSIIGYESNVKSGGVGARYFGIGADTQYQLDQIAVNLRVV
NVSTGEILSSVNTSKTILSYEVQAGVFRFIDYQRLLEGEVGYTSNEPVMLCLMSAIETGVI
FLINDGIDRGLWDLQNKAERQNDILVKYRHMSVPPES
SEQ ID NO: 3 (>P0AEA2 (16:277); mature CsgG from E. coli K12)
CLTAPPKEAARPTLMPRAQSYKDLTHLPAPTGKIFVSVYNIQDETGQFKPYPASNFSTAV
PQSATAMLVTALKDSRWFIPLERQGLQNLLNERKIIRAAQENGTVAINNRIPLQSLTAAN
IMVEGSIIGYESNVKSGGVGARYFGIGADTQYQLDQIAVNLRVVNVSTGEILSSVNTSKTI
LSYEVQAGVFRFIDYQRLLEGEVGYTSNEPVMLCLMSAIETGVIFLINDGIDRGLWDLQN
KAERQNDILVKYRHMSVPPES
SEQ ID NO: 4 (>P0AE98; coding sequence for WT CsgF from E. coli K12)
ATGCGTGTCAAACATGCAGTAGTTCTACTCATGCTTATTTCGCCATTAAGTTGGGCT
GGAACCATGACTTTCCAGTTCCGTAATCCAAACTTTGGTGGTAACCCAAATAATGGC
GCTTTTTTATTAAATAGCGCTCAGGCCCAAAACTCTTATAAAGATCCGAGCTATAAC
GATGACTTTGGTATTGAAACACCCTCAGCGTTAGATAACTTTACTCAGGCCATCCAG
TCACAAATTTTAGGTGGGCTACTGTCGAATATTAATACCGGTAAACCGGGCCGCATG
GTGACCAACGATTATATTGTCGATATTGCCAACCGCGATGGTCAATTGCAGTTGAAC
GTGACAGATCGTAAAACCGGACAAACCTCGACCATCCAGGTTTCGGGTTTACAAAA
TAACTCAACCGATTTT
SEQ ID NO: 5 (>P0AE98 (1:138); WT Pro-CsgF from E. coli K12)
MRVKHAVVLLMLISPLSWAGTMTFQFRNPNFGGNPNNGAFLLNSAQAQNSYKDPSYN
DDFGIETPSALDNFTQAIQSQILGGLLSNINTGKPGRMVTNDYIVDIANRDGQLQLNVTD
RKTGQTSTIQVSGLQNNSTDF
SEQ ID NO: 6 (>P0AE98 (20:138); WT mature CsgF from E. coli K12)
GTMTFQFRNPNFGGNPNNGAFLLNSAQAQNSYKDPSYNDDFGIETPSALDNFTQAIQSQI
LGGLLSNINTGKPGRMVTNDYIVDIANRDGQLQLNVTDRKTGQTSTIQVSGLQNNSTDF

The following Examples illustrate the invention. It is to be understood that although particular embodiments, specific configurations as well as materials and/or molecules, have been discussed herein for engineered cells and methods according to the present invention, various changes or modifications in form and detail may be made without departing from the scope and spirit of this invention. The following examples are provided to better illustrate particular embodiments, and they should not be considered limiting the application. The application is limited only by the claims.

### Example 1

Detailed methods for making and testing mutant CsgG pores and mutant CsgG/CsgF complexes are described in WO 2016/034591, WO 2017/149316, WO 2017/149317, WO 2017/149318, WO 2018/211241, and WO 2019/002893 (all incorporated by reference herein in their entirety).

E coli CsgG Pore Production

Recombinant expression vectors encoding the CsgG variant nanopores with a C-terminal Strep affinity tag and ampicillin resistance gene were transformed into chemically competent E. coli cells. The cells were plated onto an LB Agar plate containing appropriate antibiotics for selection. A single colony from the agar plate was inoculated in LB Media with antibiotics and grown overnight. The culture was diluted into autoinduction media plus necessary antibiotics and incubated at 18° C. for 68 hours. The cells were harvested through centrifugation before being lysed and extracted into 1× Bugbuster extraction reagent (Merck 70921) and 0.1% DDM. The pore was purified from the supernatant using affinity chromatography, heat treatment and then size exclusion chromatography, selecting for oligomeric nanopores as judged by SDS-PAGE.

### CsgG/CsgF Complex Formation Protocol

CsgG-CsgF complexes are prepared from nanopores purified as above and chemically synthesised CsgF peptides with or without a sulfonyl fluoride modification. Nanopores are buffer exchanged into a pH 7.0 buffer with reducing agents removed and incubated in a 8× molar excess of peptide to CsgG monomer for 1 hr at 25° C. Reactions are stopped with heating at 60° C. for 15 mins followed by centrifugation to remove any precipitate, DTT is added to 5 mM to prevent any further reaction.

FIG. 3: SDS PAGE Analysis—with Heating

300 ng of complex and CsgG-only pore control was added to individual 0.5 mL ProteinLoBind Eppendorf tubes (Fisher, 10316752) and made to 10 μL volume with Reaction Buffer. This was made to a final volume of 20 μL by the addition of 10 uL of 2× Laemmli buffer. Each sample was loaded in its entirety onto a 4-20% TGX gel (BioRad, 5671093) running with 1×TGS buffer (Sigma, T7777). This was run for 21 minutes at 300V. To image the gel, Spyro Ruby (Merk, S4942) stain was used as per the manufacturer's instructions. This was then imaged on a GE Typhoon gel imager using a 450 nm laser.

### FIGS. 4-14: DNA Squiggle (i.e., DNA Translocation Current Trace)

Electrical measurements were acquired from CsgG-only and CsgG/CsgF complexes that were inserted into MinION flow cells. After a single pore inserted into the block co-polymer membrane, 1 mL of a buffer comprising 25 mM Potassium Phosphate, 150 mM Potassium Ferrocyanide (II), 150 mM Potassium Ferricyanide (III), pH 8.0 was flowed through the system to remove any excess nanopores.

A Y-adapter is prepared by annealing DNA oligonucleotides as described previously (WO 2016/034591, which is incorporated herein in its entirety). A DNA motor was loaded and closed on the adapter. The subsequent material was HPLC purified. The Y-adapter contains a 30 C3 leader section for easier capture by the nanopore and a side arm for tethering to the membrane.

The analyte being used to assess the DNA squiggle was a 3.6-kilobase DNA section from the 3′ end of the lambda genome. Preparation of the analyte, ligating the analyte to the Y-adapter, SPRI-bead clean-up of the ligated analyte and addition to a minION flow cell was carried out using the Oxford Nanopore Technologies Q-SQK-LSK109 protocol.

Electrical measurements were acquired using minION Mk1b from Oxford Nanopore Technologies. A standard sequencing script at −180 mV was run for 2-6 hours, with static flicks every 5 minute to remove extended nanopore blocks. Raw data was collected in a bulk FAST5 file using MinKNOW software (Oxford Nanopore Technologies). A minimum of 150 pores per flow cell were tested per pore type.

Summary of data shown in FIG. 14:

| | | Median | Median |
|---|---|---|---|
| Pore monomer | Median | Range | noise |
| (conjugate) | SNR | (pA) | (pA) |
| CsgG-WT-F56Q | 6.6371 | 25.1355 | 3.7477 |
| CsgG-WT-F56Q/CsgF-(WT- | 6.6980 | 13.2280 | 1.9401 |
| del(S31-F119) | | | |
| CsgG-WT-F56Q/CsgF-(WT- | 6.9065 | 13.6694 | 1.9511 |
| K30-CH2PH-p-SO2F- | | | |
| del(S31-F119) | | | |
| CsgG-WT-F56Q/CsgF-(WT- | 6.9732 | 13.3022 | 1.8855 |
| K30-META-OSO2F-del(S31- | | | |
| F119) | | | |
| CsgG-WT-F56Q/CsgF-(WT- | 6.9125 | 13.4323 | 1.9251 |
| K30-PARA-SO2F-del(S31- | | | |
| F119) | | | |
| CsgG-WT-F56Q/CsgF-(WT- | 6.7891 | 13.1254 | 1.9147 |
| K30-META-SO2F-del(S31- | | | |
| F119) | | | |

### Synthesis of Arylsulfonyl Fluoride Containing Peptides (B. C, D and E in FIG. 2)

CsgF (NH₂-GTMTFQFRNPNFGGNPNNGAFLLNSAQAQK-CONH₂) (SEQ ID NO: 7; K denotes Lys containing sulfonyl fluoride)

The peptide was synthesized on Rink Amide ChemMatrix resin (0.48 mmol/g, 0.1 mmol scale) using automated microwave peptide synthesizer (Biotage Alstra+Initiator). Standard Fmoc solid phase peptide synthesis protocol was employed except Fmoc-Lys(Mmt)-OH was used for Lys and Boc-Gly-OH was used for N-terminal Gly. The deprotection step was carried out for 5 min at 70° C. with 20% 4-methylpiperidine in DMF (4.5 mL) and each coupling step was done for 5 min at 75° C. with Fmoc-protected amino acids (5 eq), HCTU (4.98 eq), and DIPEA (10 eq) in DMF. After completion of synthesis, Mmt protecting group was selectively removed by treatment of resin for 5 min with a solution of AcOH:TFE:DCM (1:2:7) (5 mL) and this process was repeated 4 times. After washing with DCM, arylsulfonyl fluoride group was introduced according to the literature protocol (Hoppmann, C.; Wang, L., Proximity-enabled bioreactivity to generate covalent peptide inhibitors of p53-Mdm4. Chem Commun (Camb) 2016, 52 (29), 5140-3). Briefly, the resin was treated with arylsulfonyl fluoride containing carboxylic acid (5 eq) in DMF (2.5 mL), followed by PyBOP solution in DMF (5 eq) and DIPEA (10 eq) at rt and mixed for 30 minutes followed by washing with DMF. Final deprotection and cleavage of the peptides from the resin was done using TFA/H₂O/TIS (95/2.5/2.5, v/v). After evaporating off TFA by a stream of nitrogen, crude peptides were precipitated by the addition of cold diethyl ether and purified on a reversed-phase C4 column (Vydac). Composition and purity of the peptides was confirmed by MALDI-TOF and analytical HPLC (Phenomenex Jupiter 5 um C18, 4.6×259 mm, 5 to 100% B over 20 min, flow rate: 1 mL/min).

CsgF-K30-META-SO2F-del(S31-F119) observed m/z 3426.98, calculated m/z 3427.70

CsgF-K30-PARA-SO2F-del(S31-F119) observed m/z 3427.76, calculated m/z 3427.70

CsgF-K30-CH2-PARA-SO2F-del(S31-F119) observed m/z 3442.6, calculated m/z 3442.7

### Synthesis of Arylfluorosulfate Containing Peptide a in FIG. 2

CsgF (NH₂-GTMTFQFRNPNFGGNPNNGAFLLNSAQAQK-CONH₂)) (SEQ ID NO: 7; K denotes Lys containing sulfonyl fluoride) The peptide was synthesized on Rink Amide ChemMatrix resin (0.48 mmol/g, 0.1 mmol scale) using automated microwave peptide synthesizer (Biotage Alstra+Initiator). Standard Fmoc solid phase peptide synthesis protocol was employed except Fmoc-Lys(Mmt)-OH was used for Lys and Boc-Gly-OH was used for N-terminal Gly. The deprotection step was carried out for 5 min at 70° C. with 20% 4-methylpiperidine in DMF (4.5 mL) and each coupling step was done for 5 min at 75° C. with Fmoc-protected amino acids (5 eq), HCTU (4.98 eq), and DIPEA (10 eq) in DMF. After completion of synthesis, Mmt protecting group was selectively removed by treatment of resin for 5 min with a solution of AcOH:TFE:DCM (1:2:7) (5 mL) and this process was repeated 4 times. Fluorosulfate group introduction was also performed according to the literature protocol (Baggio, C.; Udompholkul, P.; Gambini, L.; Salem, A. F.; Jossart, J.; Perry, J. J. P.; Pellecchia, M., Aryl-fluorosulfate-based Lysine Covalent Pan-Inhibitors of Apoptosis Protein (IAP) Antagonists with Cellular Efficacy. J Med Chem 2019, 62 (20), 9188-9200. Gambini, L.; Baggio, C.; Udompholkul, P.; Jossart, J.; Salem, A. F.; Perry, J. J. P.; Pellecchia, M., Covalent Inhibitors of Protein-Protein Interactions Targeting Lysine, Tyrosine, or Histidine Residues. J Med Chem 2019, 62 (11), 5616-5627). Briefly, after Mmt group removal with AcOH:TFE:DCM (1:2:7), the resin was treated with a mixture of 3-hydroxybenzoic acid (10 eq), HCTU (9.8 eq) and DIPEA (20 eq) in DMF (5 mL) for 12 h. After washing with DMF followed by DCM, the resin was treated with AISF (5 eq) and DBU (11 eq) in DCM (5 mL) overnight. Final deprotection and cleavage of the peptides from the resin was done using TFA/H2O/TIS (95/2.5/2.5, v/v). After blowing off TFA by a stream of nitrogen, crude peptides were precipitated by the addition of cold diethyl ether and purified on a reversed-phase C4 column (Vydac). Composition and purity of the peptides was confirmed by MALDI-TOF and analytical HPLC (Phenomenex Jupiter 5 um C18, 4.6×259 mm, 5 to 100% B over 20 min, flow rate: 1 mL/min).

CsgF-K30-META-OSO2F-del(S31-F119) observed m/z 3444.38, calculated m/z 3443.70

### Example 2

In this Example, 3-SO₂F is the same as META-SO₂F, 4-SO₂F is the same as PARA-SO₂F, and 4-CH₂SO₂F is the same as CH₂PH—P—SO₂F.

### Introduction

Proximity labeling has emerged as a powerful tool for probing molecular interactions and drug design. This approach relies on non-covalent binding interactions to position a molecule bearing reactive groups in close proximity to a second molecule. Because the high local concentration greatly enhances the rate of the reaction, relatively non-reactive moieties can be used to assure the reaction occurs with pinpoint regioselectivity and minimal background reactivity with solvent. Early examples such as aspirin were discovered by serendipity, but chemists now use a wide pallet of approaches to covalent labeling for the purposeful design of drugs or to probe protein interactions. Sulfonyl fluorides, which were first introduced by Roberta Coleman have proven particularly useful in this context, and sulfonyl fluorides and fluorosulfonates are now widely used as small molecule probes in chemical biology. These SuFEx groups have low reactivity towards water, but react with nucleophilic sidechains (particularly Tyr and Lys), when held in close proximity through non-covalent binding interactions with other portions of the molecule. Peptides and proteins can also be modified as active site-directed reagents or molecular probes. For example, Powers and Kettner first introduced irreversible probes such as chloromethyl ketones as well as reversible covalent probes such as boronic acids to enable potent and selective inhibition of proteases. Lei Wang and others expanded the use of reactive proteins for proximity labeling by engineering the biosynthetic machinery of E. coli to incorporate unnatural amino acids containing sulfonyl fluorides and benzylic fluorides into proteins, at once providing reagents to probe even transient protein-protein interactions as well as a new class of protein drugs. Also, Fujimori and coworkers developed methods to introduction sulfonyl fluorides into peptides as inhibitors of PhD domains in proteins. Proximity labeling within non-covalent assemblies has also been used to probe scientific problems such as the origin of life. However, despite the abundance of successful applications in fundamental research and drug design, we are unaware of proximity labeling to direct the formation of covalently crosslinked protein assemblies for practical applications in nanotechnology and engineering.

Here, we use proximity chemistry to stabilize an 18-subunit 300 kDa membrane protein complex with potential use in DNA sequencing. These pore-forming membrane protein complexes have considerable potential from single-model detection of small molecules in biology, to nucleic acid sequencing applications in nanotechnology. Specifically, this protein complex is based on the CsgG nonameric channel (FIG. 17A-B), which is part of the curli biogenesis system and is utilized for both research and translational applications. In this method a motor protein feeds a single strand of DNA through the channel. As the DNA translocates through the pore, it modulates the electrically detected ion conductance in a sequence-specific manner. Together with its natural binding partner, CsgF, the CsgG:CsgF complex forms the core apparatus of the curli secretion and assembly channel, and is comprised of 18 proteins (9xCsgG+9xCsgF). The N-terminal region of CsgF is highly conserved and critical for complex formation. The truncation of this region of CsgF to a 30-35 mer peptide results in the formation of a complex that recapitulates the original contacts with CsgG, however, as it is truncated, it becomes substantially less stable, owing to the resulting non-structured C-term region of the peptide (e. g. the CsgG:CsgF₍₁₋₃₅₎ complex is more stable than CsgG:CsgF₍₁₋₃₀₎ [Remaut, Nature Biotech 2020]). The addition of the CsgF subunits to the CsgG pore would appear particularly advantageous for DNA sequencing because it extends the extends the length and chemical composition of the pore constriction, extending attractive possibilities for greater fidelity in nucleic acid sequencing as well as increasing the pallet for new sensing applications [Remaut, Nature Biotech 2020]. However, the development of derivatives of the CsgG:CsgF complex for practical applications has been challenged by problems associated with robust assembly in vitro.

The covalent connection of CsgG and CsgF subunits would provide an attractive approach to increase stability and allow modular assembly of sensing modules. Nature's approach to proximity ligation involves disulfide formation, where non-covalent forces bring two thiols in close proximity for an oxidative coupling reaction. However, disulfides are not stable to the reducing conditions that are often used in protein assays, and it would also be helpful to avoid reliance on the use of Cys residues that might provide additional possibilities for orthogonal biocompatible reactions.

To address these limitations, we devised SuTides—sulfonyl fluoride decorated CsgF peptide derivatives, which react completely with CsgG via proximity-enhanced ligation. Given the variety of amino acid side chains these probes can interact with, the design of the SuTides, including their precise position in CsgF and choice of specific probe utilized, requires pin-point accuracy. A very high efficiency is imperative to the formation of a robust CsgG/CsgF complex, as even cases where the reaction is 95% complete would result in about half of the total CsgG pores having only 8 CsgF modifications, leading to instability of the complex and heterogeneity of channel currents (based on the multinomial probability distribution). Thus, the demands on the accuracy of the design approach are extremely high.

Nevertheless, we succeeded in the design of SuTides that react with the CsgG subunits in essentially quantitative yields. The resulting covalent complexes are highly stable, and capable of inserting into bilayers with significantly greater yields than the corresponding non-covalent complex, resulting in a 2-fold increase in the proportion of bilayer embedded stable covalent complexes, over the non-covalent complex. These findings illustrate the potential of our design approach in enabling proximity-enhanced ligation for precise, high-yield construction of high molecular weight protein complexes. Finally, we employed molecular dynamics to probe the high efficiency of the ligation, resulting in insights that might be helpful in future applications of SuFEx chemistry in a variety of molecular contexts.

### Results

### Design and Synthesis

For efficient SuFEx labeling, we chose a phenylsulfonyl fluoride derivative of lysine, which was chosen based on previous random crosslinking studies that showed this linkage provided a compromise between flexibility and reactivity. To form a stable complex between CsgG and the CsgF peptide we began by identifying positions to place the probe in distances and angles conducive of proximity enhanced ligation between the probe and a target nucleophilic residue (FIG. 17C). Moreover, the introduced crosslink should retain the native structure of the protein and not occlude the ion-conducting pore. This requirement eliminated many potential positions of the target nucleophilic amino acid sidechains in CsgG and sulfonyl fluorides in the CsgF:CsgG complex. We chose Tyr196 on CsgG as the target nucleophile, and the six C-terminal residues of the CsgF peptide as possible positions for introduction of the sulfonyl fluoride warhead. The Cₐₗₚₕₐ-Cₐₗₚₕₐ distances between these residues and Tyr196 are all within the 5-10 Å range (FIG. 17D), which has been suggested as a rough guideline for the optimal distance for sulfonyl fluoride-based proximity enhanced ligation. A manual rotamer analysis, considering distances and angles between the hydroxyl oxygen on Tyr196 and the sulfonyl sulfur of each probe, on each of the six C-terminal residues of the CsgF₍₁₋₃₅₎ peptide, identified position 30 as the preferred location for placement of the warhead.

Three SuTides were synthesized by incorporating each of the different probes in position 30 of the sequence of WT CsgF₍₁₋₂₉₎. Each introduces a Lys residue at position 30, to which a 3 or 4-substituted sulfonyl-phenyl fluoride or 4-sulfonyl-benzyl fluoride was introduced via an amide bond to the primary amine of K30 (termed 3-SO₂F-CsgF, 4-SO₂F-CsgF and 4-CH₂SO₂F-CsgF, respectively) (FIG. 17E). The peptides were synthesized by solid phase peptide synthesis with a trityl protecting group on the C-terminal Lys, which was removed following completion of the chain assembly. The trityl was then deprotected, and the resulting Lys30 amine was coupled to the appropriate carboxylic acids containing the sulfonyl fluorides completed. The remaining protecting groups and concomitant removal from the resin was carried out by treatment with trifluoracetic, and the resulting SuTides were purified to homogeneity. We observed no problems associated with the stability or hydrolysis of the SuTides in acidic aqueous solution or when stored in DMSO at −20° C.

Reaction of SuTides with CsgG

Each of the SuTides were found to react in nearly quantitative yield with preformed nonameric CsgG pore complexes when they were reacted in 8-fold molar excess (over CsgG monomers) overnight (FIG. 18A). We used mass spectrometry of a tryptic digest of the resulting products to confirm the covalent adduct (data not shown). The intensity of the peptide that houses the targeted Tyr196 (CsgG 191-198) decreased by 10 to 100-fold when compared to the WT CsgG:CsgF complex (data not shown). This peptide (FIDYQR) also lacks other residues that can easily react with sulfonyl-fluorides confirming attachment to the targeted Tyr residue. Finally, intensities of other surrounding peptide fragments that are rich in Lys residues (that are highly reactive with sulfonyl fluorides) were unaffected before and after trypsin treatment (data not shown). Together, these results demonstrate the regioselectivity of the reaction.

The time course of the reaction of each SuTide with CsgG were evaluated by sampling time points via SDS-PAGE (FIG. 18A). SuTide 3-SO₂F-CsgF reacted with a halftime (t_{1/2}) of 0.66 hr; t_{1/2} for 4-SO₂F-CsgF was 1.5 hr and t_{1/2} for 4-CH₂SO₂F-CsgF was 4 hr (corresponding to first order rate constants of approximately k₁=1, 0.46 and 0.17 hr⁻¹, respectively) (FIG. 18B). These differences likely reflect contributions from both the intrinsic reactivity as well as the effective concentration of the reacting groups. To help dissect these effects we measured the reaction kinetics of acetyl-Tyr-O-methyl ester (Ac-Tyr-OMe) with the n-butylamide version of 3-SO₂F-CsgF (3-SO₂F-But). Under a large excess of the sulfonyl fluoride (5.0 mM 3-SO₂F-But, 0.5 mM Ac-Tyr-OMe) we observed a pseudo first order rate constant of 0.01 hr⁻¹ for the reaction of Ac-Tyr-Ome (data not shown). The corresponding second order rate constant, k₂, is 2 M⁻¹ hr⁻¹ if the concentration of 3-SO₂F-But is considered, providing a good metric of the reactivity of the 3-SO₂F-warhead. The ratio of the first order rate constant for reaction of 3-SO₂F-CsgF in complex with CsgG to the second order rate constant for the model reaction with 3-SO₂F-But (k₁/k₂) gives an effective concentration of 0.5 M.

While we did not determine rates for the corresponding models of 4-SO₂F-CsgF and 4-CH₂SO₂F-CsgF, an extensive comparison of the effects of these substituents and substitution patterns on the relative rates of reactivity of sulfonyl fluorides with Ac-Tyr-OMe are available from [Gilbert et al. ACS Chem. Bio 2023]. Linear scaling as in [Gilbert et al. ACS Chem. Bio 2023] provides approximate calculations of the second order rate constants as 2.4 M⁻¹ hr⁻¹ and 0.6 M⁻¹ hr⁻¹, allowing one to account for differences in the intrinsic chemical reactivity of warheads associated with the three probes. The corresponding C_{eff} computed from these values and the pseudo-first order rate constants for 4-SO₂F-CsgF and 4-CH₂SO₂F-CsgF were 0.19 M and 0.28 M.

### Molecular Dynamics

Having accounted for differences in chemical reactivity we next used molecular dynamics (MD) simulations to provide a qualitative comparison of the kinetically defined C_{eff} with that expected from the dynamic ensemble of conformers observed in the CsgG-3-SO₂F-CsgF complex. Given that the sulfonyl-fluorides were situated on a flexible Lys residue at the C-terminus of the CsgF, we expected significant local flexibility. Thus, MD would appear well suited to provide a reasonable estimate of the fraction of time that the reacting groups were in van der Waals contact and at an angle conducive to the displacement reaction. Clearly, more sophisticated calculations would be required to determine absolute rates, but we expected that classical MD simulations might be able to provide insight into the origin of the high effective concentration in the pre-reacting complex. All-atom simulations were conducted with a simulated temperature of 293K and the AMBER force field. Three independent 200 nsec simulations of the nonameric CsgG-SuTide complexes were computed, corresponding to a total simulation time of 3*9*200=5,400 nsec assuring good sampling on the high nsec to low microsecond times scale. Examples of the CsgG Tyr196 and 3-SO₂F-CsgF in the top cluster is shown in FIG. 19A. Moreover, distances between the phenol oxygen of the Tyr and the sulfonyl S (d_{O,S}) ranged between 3.1 to app. 20 Å, indicating good sampling over a wide range of distances. We next constructed radial distribution plots of d_{O,S} to determine the probability of finding the reactive phenolic O within van der Waals distance of the sulfonyl-fluoride's sulfur atom (FIG. 19B).

These distributions were next computed to probabilities per unit volume from which apparent Molar concentrations (Cₐₚₚ) were computed. The value of Cₐₚₚ in the distance bin corresponding to close van der Waals contact (<4 Å) was computed to be 1.3 M for 3-SO₂F-CsgF; the corresponding values were 0.5 M and 1.1 M for 4-SO₂F-CsgF and 4-CH₂SO₂F-CsgF, respectively (FIG. 19C). These values are within a factor of three of one another, and agree in rank order. We next examined the angle, q_{O,S,F} between the incoming phenolic oxygen relative to the fluoride leaving group as a function of d_{O,S}. The nucleophile approaches trans to the fluoride in SuFEx reactions. Thus, a value of q_{O,S,F} between approximately 140° and 180° paired with a value of d_{O,S}<4 Å would be expected to facilitate the reaction (FIG. 19D). Indeed, all three SuTides showed maxima in 2-dimensional plots of d_{O,S} versus q_{O,S,F}, and which was highest for the most reactive peptide, 3-SO₂F-CsgF (FIG. 19E).

### Discussion and Conclusion

A fundamental objective of this study was to develop methods to enhance the assembly, stability and yield of very large protein complexes. The use of sulfonyl fluorides for this purpose has solved a practical problem associated with the assembly of nanopores with tailored pore lining, which can now be systematically varied to maximize the fidelity of DNA sequencing. Moreover, this approach provides a useful method for introduction of a variety of proteins or peptides for single-molecule detection and a potential platform for protein sequencing.

A few recent efforts concerning the synthesis of sulfonyl fluorides to enhance the affinity and stability of peptide-protein complexes have been reported. However, the placement of the warhead was empirically placed to maximize the yield of protein-peptide complexes. In this case, we drew on a more systematic search of sequence positions and rotamers, and ultimately, the successful placement relied largely on our understanding of protein structure and chemical reactivity. However, we also wondered whether molecular dynamics calculations might provide insight into the high C_{eff} and regioselectivity seen here. We were pleasantly surprised to see a reasonable absolute and rank order agreement between Cₐₚₚ and C_{eff}. Consideration of the distances as well as angles of approach of the nucleophilic sidechains was in good agreement with the observed reactivity. We expect that this approach will be useful in cases where there is significant flexibility between the reacting groups, in which case MD can be expected to guide design of proximal positions that can lead to high reactivity. However, in cases where the nucleophile and warhead are less flexible, we expect that stereo-electronic effects will become more dominant and QM/MM calculations would be needed to accurately rank potential designs. While the work reported here is focused on sulfonyl fluorides, a variety of additional chemistries, including benzylic fluorides could potentially be advantageous to explore.

1. A pore monomer conjugate comprising a CsgG pore monomer attached to a CsgF peptide, wherein the CsgF peptide is attached to the CsgG pore monomer by a sulfonyl fluoride-containing linker, a sulfonyl triazole-containing linker, a fluorosulfate-containing linker, or fluoroacetamide-containing linker.

2. A pore monomer conjugate according to claim 1, wherein (a) the N terminus or the residue at any one of positions 1 to 40 in the CsgF peptide is attached to the CsgG pore monomer by the linker or (b) the residue at position 30 of the CsgF peptide or the residue in the CsgF peptide at the position corresponding to position 30 in SEQ ID NO: 6 is attached to the CsgG pore monomer by the linker.

3. A pore monomer conjugate according to claim 1 or 2, wherein the CsgF peptide is attached to a residue in the loop forming regions of the CsgG pore monomer.

4. A pore monomer conjugate according to claim 3, wherein the loop forming regions correspond to positions 142-146 and 190-200 in SEQ ID NO: 3.

5. A pore monomer conjugate according to any one of the preceding claims, wherein the CsgF peptide is attached to a serine, lysine, threonine, tyrosine, histidine, or cysteine residue in the CsgG pore monomer by the linker.

6. A pore monomer conjugate according to any one of the preceding claims, wherein the CsgF peptide is covalently attached to the CsgG pore monomer by the linker.

7. A pore monomer conjugate comprising a CsgG pore monomer attached to a CsgF peptide, wherein the CsgF peptide is attached to a residue in the CsgG pore monomer corresponding to any one of positions 143, 146, 190, 192, 193, 194, 195, 196, 197, 198, 199 or 200 in SEQ ID NO: 3.

8. A pore monomer conjugate according to claim 7, wherein the residue is a serine, lysine, threonine, tyrosine, histidine, or cysteine residue in the CsgG pore monomer and/or is modified with a reactive group.

9. A pore monomer conjugate according to claim 7 or 8, wherein the CsgF peptide is attached to the CsgG pore monomer using (a) an amine-reactive group, such as a thioester, NHS-ester, pentafluorophenyl ester, benzylic halide, benzylic fluoride, sulfonyl halide, sulfonyl fluoride, halosulfate, fluorosulfate, sulfonyl triazole, or boronic acid or (b) an oxygen-reactive group, such as an alkyl halide, alkyl fluoride, sulfonyl halide, sulfonyl fluoride, halosulfate, fluorosulfate, or sulfonyl triazoles.

10. A pore monomer conjugate according to any one of claims 7-9, wherein the residue in the CsgF peptide defined in claim 2 is attached to the CsgG pore monomer.

11. A pore monomer conjugate according to any one of the preceding claims, wherein the CsgG pore monomer is a variant of SEQ ID NO: 3 and/or the CsgF peptide is a variant of SEQ ID NO: 6.

12. A construct comprising two or more covalently attached pore monomer conjugates according to any one of claims 1-11.

13. A construct according to claim 12, wherein the pore monomer conjugates are genetically fused and/or are attached via a linker.

14. A pore complex comprising at least one pore monomer conjugate according to any one of the preceding claims or at least one construct according to claim 12 or 13, wherein the CsgF peptide(s) form(s) a constriction in the pore complex.

15. A pore complex according to claim 14, wherein the pore complex is a homooligomer comprising 6 to 10 pore monomer conjugates according to any one of claims 1-11 or 1-5 constructs according to claim 12 or 13.

16. A pore complex according to claim 14 or 15, wherein the CsgF peptide(s) is/are inserted into the lumen of the pore complex.

17. A pore multimer comprising two or more pores, wherein at least one of the pores is a pore complex according to any one of claims 14-16.

18. A pore complex according to any one of claims 14-16 or a pore multimer according to claim 17, which is comprised in a membrane.

19. A membrane comprising a pore complex according to any one of claims 14-16 or a pore multimer according to claim 17.

20. A method for producing a pore monomer conjugate according to any one of claims 1-11 comprising attaching the CsgF peptide to the CsgG pore monomer.

21. A method for producing a pore complex according to any one of claims 14-16 or a pore multimer according to claim 17, the method comprising expressing at least one pore monomer conjugate according to any one of claims 1-11 or a construct according to claim 12 or 13 and sufficient pore monomers or constructs to form the pore complex or the pore multimer in a host cell and allowing the pore complex or the pore multimer to form in the host cell.

22. A method for producing a pore complex according to any one of claims 14-16 or a pore multimer according to claim 17, the method comprising contacting at least one pore monomer conjugate according to any one of claims 1-11 or a construct according to claim 12 or 13 with sufficient pore monomers or constructs in vitro and allowing the formation of the pore complex or the pore multimer.

23. A method for determining the presence, absence or one or more characteristics of a target analyte, comprising the steps of:
(i) contacting the target analyte with a pore complex according to any one of claims 14-16 or a pore multimer according to claim 17, such that the target analyte moves with respect to the pore complex or the pore multimer; and
(ii) taking one or more measurements as the analyte moves with respect to the pore complex or the pore multimer and thereby determining the presence, absence or one or more characteristics of the analyte.

24. A method according to claim 23, wherein the analyte is a peptide, a polypeptide, a polysaccharide, a small organic or inorganic compound, such as pharmacologically active compounds, toxic compounds, and pollutants.

25. A method according to claim 24, wherein the analyte is a polynucleotide.

26. A method according to claim 25, wherein the polynucleotide comprises at least one homopolymeric region.

27. A method according to claim 25 or 26, comprising determining one or more characteristics selected from (i) the length of the polynucleotide, (ii) the identity of the polynucleotide, (iii) the sequence of the polynucleotide, (iv) the secondary structure of the polynucleotide and (v) whether or not the polynucleotide is modified.

28. A method of characterising a polynucleotide, a peptide or a polypeptide using a pore complex according to any one of claims 14-16 or a pore multimer according to claim 17.

29. Use of a pore complex according to any one of claims 14-16 or a pore multimer according to claim 17 to determine the presence, absence or one or more characteristics of a target analyte.

30. A polynucleotide which encodes a pore monomer conjugate according to any one of claims 1-11 or a construct according to claim 12 or 13.

31. A kit for characterising a target analyte comprising (a) a pore complex according to any one of claims 14-16 or a pore multimer according to claim 17 and (b) the components of a membrane.

32. A kit for characterising a target polynucleotide or a target polypeptide comprising (a) a pore complex according to any one of claims 14-16 or a pore multimer according to claim 17 and (b) a polynucleotide binding protein or a polypeptide handling enzyme.

33. An apparatus for characterising a target polynucleotide or a target polypeptide in a sample, comprising (a) a plurality of pore complexes according to any one of claims 14-16 or a plurality of pore multimers according to claim 17 and (b) a plurality of polynucleotide binding proteins or a plurality of polypeptide handling enzymes.

34. An array comprising a plurality of membranes according to claim 19.

35. A system comprising (a) a membrane according to claim 19 or an array according to claim 34, (b) means for applying a potential across the membrane(s) and (c) means for detecting electrical or optical signals across the membrane(s).

36. An apparatus comprising a pore complex according to any one of claims 14-16 or a pore multimer according to claim 17 inserted into an in vitro membrane.

37. An apparatus produced by a method comprising (i) obtaining a pore complex according to any one of claims 14-16 or a pore multimer according to claim 17 and (ii) contacting the pore complex or a pore multimer with an in vitro membrane such that the pore complex or the pore multimer is inserted in the in vitro membrane.


SEQUENCE LISTING (7 sequences)

SEQ ID NO: 1 - DNA, 834 bp, Escherichia coli
  source 1..834: mol_type = genomic DNA; note = K-12
atgcagcgcttatttcttttggttgccgtcatgttactgagcggatgcttaaccgccccgcctaaagaagccgccagaccgacattaatgcctcgtgctcagagctacaaagatttgacccatctgccagcgccgacgggtaaaatctttgtttcggtatacaacattcaggacgaaaccgggcaatttaaaccctacccggcaagtaacttctccactgctgttccgcaaagcgccacggcaatgctggtcacggcactgaaagattctcgctggtttataccgctggagcgccagggcttacaaaacctgcttaacgagcgcaagattattcgtgcggcacaagaaaacggcacggttgccattaataaccgaatcccgctgcaatctttaacggcggcaaatatcatggttgaaggttcgattatcggttatgaaagcaacgtcaaatctggcggggttggggcaagatattttggcatcggtgccgacacgcaataccagctcgatcagattgccgtgaacctgcgcgtcgtcaatgtgagtaccggcgagatcctttcttcggtgaacaccagtaagacgatactttcctatgaagttcaggccggggttttccgctttattgactaccagcgcttgcttgaaggggaagtgggttacacctcgaacgaacctgttatgctgtgcctgatgtcggctatcgaaacaggggtcattttcctgattaatgatggtatcgaccgtggtctgtgggatttgcaaaataaagcagaacggcagaatgacattctggtgaaataccgccatatgtcggttccaccggaatcctga

SEQ ID NO: 2 - protein, 277 aa, Escherichia coli
  source 1..277: note = K-12
MQRLFLLVAVMLLSGCLTAPPKEAARPTLMPRAQSYKDLTHLPAPTGKIFVSVYNIQDETGQFKPYPASNFSTAVPQSATAMLVTALKDSRWFIPLERQGLQNLLNERKIIRAAQENGTVAINNRIPLQSLTAANIMVEGSIIGYESNVKSGGVGARYFGIGADTQYQLDQIAVNLRVVNVSTGEILSSVNTSKTILSYEVQAGVFRFIDYQRLLEGEVGYTSNEPVMLCLMSAIETGVIFLINDGIDRGLWDLQNKAERQNDILVKYRHMSVPPES

SEQ ID NO: 3 - protein, 262 aa, Escherichia coli
  source 1..262: note = K-12
CLTAPPKEAARPTLMPRAQSYKDLTHLPAPTGKIFVSVYNIQDETGQFKPYPASNFSTAVPQSATAMLVTALKDSRWFIPLERQGLQNLLNERKIIRAAQENGTVAINNRIPLQSLTAANIMVEGSIIGYESNVKSGGVGARYFGIGADTQYQLDQIAVNLRVVNVSTGEILSSVNTSKTILSYEVQAGVFRFIDYQRLLEGEVGYTSNEPVMLCLMSAIETGVIFLINDGIDRGLWDLQNKAERQNDILVKYRHMSVPPES

SEQ ID NO: 4 - DNA, 414 bp, Escherichia coli
  source 1..414: mol_type = genomic DNA; note = K-12
atgcgtgtcaaacatgcagtagttctactcatgcttatttcgccattaagttgggctggaaccatgactttccagttccgtaatccaaactttggtggtaacccaaataatggcgcttttttattaaatagcgctcaggcccaaaactcttataaagatccgagctataacgatgactttggtattgaaacaccctcagcgttagataactttactcaggccatccagtcacaaattttaggtgggctactgtcgaatattaataccggtaaaccgggccgcatggtgaccaacgattatattgtcgatattgccaaccgcgatggtcaattgcagttgaacgtgacagatcgtaaaaccggacaaacctcgaccatccaggtttcgggtttacaaaataactcaaccgatttt

SEQ ID NO: 5 - protein, 138 aa, Escherichia coli
  source 1..138: note = K-12
MRVKHAVVLLMLISPLSWAGTMTFQFRNPNFGGNPNNGAFLLNSAQAQNSYKDPSYNDDFGIETPSALDNFTQAIQSQILGGLLSNINTGKPGRMVTNDYIVDIANRDGQLQLNVTDRKTGQTSTIQVSGLQNNSTDF

SEQ ID NO: 6 - protein, 119 aa, Escherichia coli
  source 1..119: note = K-12
GTMTFQFRNPNFGGNPNNGAFLLNSAQAQNSYKDPSYNDDFGIETPSALDNFTQAIQSQILGGLLSNINTGKPGRMVTNDYIVDIANRDGQLQLNVTDRKTGQTSTIQVSGLQNNSTDF

SEQ ID NO: 7 - protein, 30 aa, synthetic construct
  MOD_RES 30: note = lysine containing sulfonyl fluoride
GTMTFQFRNPNFGGNPNNGAFLLNSAQAQK